(19) United States
(12) Patent Application Publication
(10) Pub. No.: US 2025/0222269 A1
(43) Pub. Date: Jul. 10, 2025

(54) METHODS AND DEVICES FOR ELECTROMAGNETIC ENERGY DELIVERY FOR BIOLOGICAL TISSUE TREATMENT

(71) Applicant: Like Zeng, Pleasanton, CA (US)

(72) Inventor: Like Zeng, Pleasanton, CA (US)

(21) Appl. No.: 19/016,842

(22) Filed: Jan. 10, 2025

Related U.S. Application Data
(63) Continuation of application No. PCT/US2023/070037, filed on Jul. 12, 2023.
(60) Provisional application No. 63/388,601, filed on Jul. 12, 2022.

(51) Int. Cl. A61N 5/06

(52) U.S. Cl. CPC A61N 5/06; A61N 2005/0627

(57) ABSTRACT

A device (40) delivers energy to biological tissue (49) during time intervals that extend over a treatment period of time. While delivering a portion of the energy in a time interval of the time intervals, the device (40) determines one or more instantaneous received power (IRP) values based on measured electrical parameters. The device (40) determines, at least once during the time interval, a power variance based at least on the one or more IRP values. The device (40) updates a duration of one or more of the time intervals based on determining that a change in the power variance satisfies a threshold tolerance level. The device (40) adjusts, during the time interval, at least one of operating condition of a generator (41) to cause an amount of the energy delivered to the biological tissue (49) to be equal or substantially equal to an amount of energy identified by a treatment energy delivery value.

### RELATED APPLICATION

This application claims priority, as a continuation application under 35 U.S.C. § 119 and/or 35 U.S.C. § 120, to PCT Application No. PCT/US2023/070037, filed Jul. 7, 2023, which claims priority to U.S. Provisional Application No. 63/388,601, filed Jul. 12, 2022, each of which is expressly incorporated by reference herein, in its entirety.

### BACKGROUND

Energy emitting devices can include electromagnetic energy generators that convert electromagnetic energy to different forms of output energy. For example, some energy emitting devices can convert electromagnetic energy to energy forms that can be absorbed by human tissue, such as radio frequency (RF) current, a RF electromagnetic field, electromagnetic radiation, ultrasound waves, energy in the form of a laser, and/or the like. This allows the energy emitting devices to heat human tissue, thereby triggering a biological reaction in the heated tissue to achieve a therapeutic effect.

One of the shortcomings of currently available energy emitting devices for treating tissue is the formation of temperature hotspots. In general, when energy (e.g., RF current, etc.) is delivered to tissue through an electrode, the energy concentrates around the edges of the electrode (and in particular, around the sharp edges). This effect is known as an edge effect. In the case of a circular disc electrode, edge effect manifests as a higher current density around the perimeter of the circular disc and has a relatively low current density in the center. For a square-shaped electrode, there is typically a high current density around the entire perimeter and an even higher current density at the corners. This concentration of RF current results in undesirable temperature hotspots, which in turn causes a patient exposed to the RF current to experience pain.

Patient pain is inherent in tissue treatments using electromagnetic energy. Patient pain is typically regulated to optimize the treatment result while also minimizing patient discomfort. To reduce the pain, a patient may be given an oral pain medication and/or a local topical anesthesia cream that can be applied as a numbing agent. These solutions significantly increase patient preparation time and cost and are cumbersome to both patients and the physicians.

As described above, higher temperatures near the peripheral edges of electrodes can create hot spots that cause the patient to experience pain. One way to combat this is to have an operator of the energy emitting device (e.g., a physician) lower an energy intensity level to counteract the temperature hotspots. This provides the patient with a more tolerable treatment experience. However, one consequence of lowering the energy intensity setting is that the patient may be undertreated and fail to receive a therapeutic amount of energy, thus compromising the overall treatment efficacy.

Many energy emitting devices use open loop power control. An operator typically selects a treatment intensity level or a treatment energy delivery level that dictates the output power provided by the electromagnetic energy generator. However, output power using a fixed operating condition is a function of load impedance. Typically, this output power-load impedance relationship can be approximated by a Gaussian curve. The output power increases as the load impedance increases, the output power reaches a maximum, and then the output power decreases as the load impedance further increases. In many treatment procedures that use energy emitting devices, the load impedance is affected by a number of factors, such as electrical properties of the patient tissue, patient movement, tissue perfusion, a coupling medium, contact pressure, and/or the like. Therefore, the load impedance experiences frequent changes throughout the treatment, leading to a varying amount of power delivered to the patient.

In a distributed system, where a coaxial cable or waveguide is used to carry the energy from the electromagnetic energy generator to the load, and when its physical length is a significant portion of the electromagnetic energy's wavelength, the load only receives maximum power when its impedance matches the characteristic impedance of the coaxial cable or waveguide. Any deviation from the characteristic impedance will lead to a decreased amount of power delivered to the load.

This dependency between load impedance and output power is not adequately addressed in energy emitting devices. The consequences are two-fold: first, the power delivered to the patient is unpredictable. Consequently, whether the patient receives the desired therapeutic level of energy may be unpredictable. The patient can be over-treated or under-treated, causing serious uncertainties on treatment efficacy. Second, when the electromagnetic energy is converted to heat in patient tissue to achieve a therapeutic effect, the change in load impedance may cause a sudden burst in the power delivered to the patient. If not managed properly, this sudden burst in delivered power can lead to rapid rise in temperature in the treated tissue. If the power delivered to the patient is too high, pain sensing nociceptors of the patient may fire, causing the patient to experience a significant amount of pain.

Some modern energy based medical devices adopt a number of sensors to provide feedback to adjust the electromagnetic energy emitting device's drive level to alleviate some of the above-mentioned concerns. However, the sensors typically are not capable of providing enough temporal and spatial resolutions. For example, electromagnetic energy delivery devices often include contact-based temperature sensors, including thermistors or thermocouples to monitor the temperature of the treated tissue. However, due to the inherent physical constructions of these contact-based temperature sensors, these sensors are unable to provide precise tissue temperature measurements. Moreover, contact-based sensors are limited by the sampling rate due to sensor stabilization requirements. Some devices use non-contact temperature sensors, such as an infrared (IR) sensor. However, these sensors can only accurately detect the temperature on the surface closest to the sensor. Furthermore, when the treatment area is covered by a coupling gel or a related material or substance, this technique is unable to efficiently and/or effectively monitor the patient's temperature.

Therefore, there is a need for a device and/or method to reduce temperature hotspots associated with tissue treatments such that patient discomfort is alleviated, and for delivering a precise amount of power to the tissue of the patient, thereby achieving more consistent therapeutic results.

### SUMMARY OF THE INVENTION

In an aspect of the invention, a method of delivering electromagnetic energy from an energy emitting device for treating biological tissue as part of a treatment procedure is disclosed. The method includes generating electromagnetic energy with a generator operating under one or more operating conditions for delivering the electromagnetic energy to the biological tissue. The method further includes delivering the electromagnetic energy through the energy emitting device to the biological tissue during a plurality of time intervals that extend over a treatment period of time that represents a duration of the treatment procedure. While delivering a portion of the electromagnetic energy in a time interval of the plurality of time intervals, the method further includes determining one or more instantaneous received power values based on measured electrical parameters. The method further includes determining, at least once during the time interval, a power variance based at least in part on the one or more instantaneous received power values. The method further includes updating a duration of one or more time intervals, of the plurality of time intervals, based on determining that a change in the power variance satisfies a threshold tolerance level. The method further includes adjusting, during the time interval, at least one of the one or more operating conditions of the generator, adjusting at least one of the one or more operating conditions causing an amount of the electromagnetic energy delivered to the biological tissue to be equal to or substantially equal to an amount of electromagnetic energy identified by a treatment energy delivery value.

In an embodiment of the invention, when determining an instantaneous received power value, of the one or more instantaneous received power values, the method includes determining the instantaneous received power value based on the difference between a measured instantaneous forward power level and a measured instantaneous reflected power level.

In another embodiment of the invention, the one or more instantaneous received power values are a plurality of instantaneous received power values. In this embodiment, when determining the power variance, the method includes determining the power variance based on a difference between a minimum instantaneous received power value and a maximum instantaneous received power value of the plurality of instantaneous received power values.

In another embodiment of the invention, the one or more instantaneous received power values include only a single instantaneous received power value determined during the time interval. In this embodiment, when determining the power variance, the method includes determining the power variance based on a difference between the single instantaneous received power value determined during the time interval and a previously determined instantaneous received power value determined during a previous time interval of the plurality of time intervals.

In another embodiment of the invention, the threshold tolerance level is an upper bound threshold tolerance level. In this embodiment, when updating the duration of the one or more time intervals, the method includes reducing the duration of the one or more time intervals based on determining that the change in the power variance exceeds the upper bound threshold tolerance level.

In another embodiment of the invention, the threshold tolerance level is a lower bound threshold tolerance level. In this embodiment, when updating the duration of the one or more time intervals, the method includes increasing the duration of the one or more time intervals based on determining that the change in the power variance is less than the lower bound threshold tolerance level.

In another embodiment of the invention, the treatment period is further defined over a set of pulse width modulation (PWM) periods. In this embodiment, the method further includes determining, at the beginning of a first time interval of the plurality of time intervals, a remaining amount of the electromagnetic energy to deliver during a first PWM period of the set of PWM periods. The method further includes determining a period of additional time needed to deliver the remaining amount of the electromagnetic energy during the first PWM period. The method further includes comparing a duration of the period of additional time and a duration of an upcoming time interval of the plurality of time intervals. The method further includes selecting an amount of time during which to deliver the remaining amount of the electromagnetic energy during the first PWM period based on comparing the duration of the period of additional time and the duration of the upcoming time interval. In this embodiment, when adjusting the one or more operating conditions, the method includes adjusting an operating condition, of the one or more operating conditions, to cause the generator to deliver, for the selected amount of time, the remaining amount of the electromagnetic energy during the first PWM period. In some embodiments, when selecting the amount of time during which to deliver the remaining amount of the electromagnetic energy over the first PWM period, the method includes selecting the period of additional time based on determining that the duration of the period of additional time does not exceed the duration of the upcoming time interval, or selecting the upcoming time interval based on based on determining that the period of additional time exceeds the duration of the upcoming time interval. In some embodiments, when determining the period of additional time needed to deliver the remaining amount of electromagnetic energy during the first PWM period, the method includes determining the period of additional time using the following equation:

$$t 1 = \frac{{E}_{r}}{P} ,$$

where t1 is the additional period of time, Eᵣ is the remaining amount of the electromagnetic energy to deliver during the first PWM period, and P is based on the one or more instantaneous received power values. P may be based on a single instantaneous received power value, multiple instantaneous received power values, an average of multiple instantaneous received power values, and/or the like, as will be further explained in the detailed description.

In another embodiment of the invention, the one or more operating conditions include at least one of: a state of the generator of the electromagnetic device, or a drive level of the generator.

In another embodiment of the invention, each respective time interval, of the one or more time intervals, have a duration between 1 nanosecond (ns) and 10 second(s), preferably between 100 microseconds (us) to 10 milliseconds (ms).

In another embodiment of the invention, the method further comprises determining that the one or more instantaneous received power values are not within a threshold range of values that represent normal operating conditions. The method further comprises performing one or more actions based on determining that the plurality of instantaneous received power values are not within the threshold range of values. The one or more actions include at least one of: pausing the treatment procedure, terminating the treatment procedure, adjusting an operating condition, of the one or more operating conditions, to reduce the amount of electromagnetic energy delivered by at least 60%, or generating and transmitting an alert signal indicating that the energy emitting device is not operating under normal operating conditions.

In another aspect of the invention, an energy emitting device is provided for treatment of biological tissue. The energy emitting device includes a generator, a power sensing unit, an electromagnetic energy delivery unit, and a processor. The generator is configured to generate electromagnetic energy and to operate under one or more operating conditions for delivering the electromagnetic energy to the biological tissue. The power sensing unit is configured to measure instantaneous power levels. The electromagnetic energy delivery unit is configured to convert the electromagnetic energy to energy forms permissible for the treatment of the biological tissue. The processor is configured to instruct the generator to generate the electromagnetic energy, the electromagnetic energy to be delivered to the biological tissue during a plurality of time intervals that extend over a treatment period of time that represents a duration of the treatment procedure. While a portion of the electromagnetic energy is delivered during a time interval of the plurality of time intervals, the processor is further configured to determine one or more instantaneous received power values based on measured electrical parameters. The processor is further configured to determine, at least once during the time interval, a power variance based at least in part on the one or more instantaneous received power values. The processor is further configured to update a duration of one or more time intervals, of the plurality of time intervals, based on determining that a change in the power variance satisfies a threshold tolerance level. The processor is further configured to adjust, during the time interval, at least one of the one or more operating conditions of the generator. Adjusting at least one of the one or more operating conditions causes an amount of the electromagnetic energy delivered to the biological tissue to be equal to or substantially equal to an amount of electromagnetic energy identified by a treatment energy delivery value.

In another embodiment of the invention, the processor, when determining an instantaneous received power value, of the one or more instantaneous received power values, is configured to determine the instantaneous received power value based on the difference between a measured instantaneous forward power level and a measured instantaneous reflected power level.

In another embodiment of the invention, the threshold tolerance level is an upper bound threshold tolerance level. In this embodiment, the processor, when updating the duration of the one or more time intervals, is configured to reduce the duration of the one or more time intervals based on determining that the change in the power variance exceeds the upper bound threshold tolerance level.

In another embodiment of the invention, the threshold tolerance level is a lower bound threshold tolerance level. In this embodiment, the processor, when updating the duration of the one or more time intervals, is configured to increase the duration of the one or more time intervals based on determining that the change in the power variance is less than the lower bound threshold tolerance level.

In another embodiment of the invention, the treatment period is further defined over a set of pulse width modulation (PWM) periods. In this embodiment, the processor is further configured to determine, at the beginning of a first time interval of the plurality of time intervals, a remaining amount of the electromagnetic energy to deliver during a first PWM period of the set of PWM periods. The processor is further configured to determine a period of additional time needed to deliver the remaining amount of the electromagnetic energy during the first PWM period. The processor is further configured to compare a duration of the period of additional time and a duration of an upcoming time interval of the plurality of time intervals. The processor is further configured to select an amount of time during which to deliver the remaining amount of the electromagnetic energy during the first PWM period based on comparing the duration of the period of additional time and the duration of the upcoming time interval. In this embodiment, when adjusting at least one of the one or more operating conditions, the processor is configured to adjust an operating condition, of the one or more operating conditions, to cause the generator to deliver, for the selected amount of time, the remaining amount of the electromagnetic energy during the first PWM period.

In another embodiment of the invention, the one or more operating conditions include at least one of: a state of the generator of the electromagnetic device, or a drive level of the generator.

In another embodiment of the invention, the processor is further configured to determine that the one or more instantaneous received power values are not within a threshold range of values that represent normal operating conditions. The processor is further configured to perform one or more actions based on determining that the plurality of instantaneous received power values are not within the threshold range of values.

In another embodiment of the invention, the power sensing unit includes two directional couplers and one bidirectional coupler.

### BRIEF DESCRIPTION OF THE DRAWINGS

FIG. 1 is a diagram illustrating a graphical representation of action potential firing patterns of nociceptors sensing noxious heat.

FIG. 2 is a diagram illustrating a representative action potential waveform.

FIG. 3 is a diagram illustrating temperature profiles associated with different pulse width modulation (PWM) frequencies.

FIG. 4 is a diagram illustrating instantaneous received power measured relative to time.

FIG. 5A is a diagram illustrating a graphical representation of an energy emitting device determining one or more instantaneous received power values over one or more time intervals.

FIG. 5B is another diagram illustrating a graphical representation of the energy emitting device determining one or more instantaneous received power values over one or more time intervals.

FIG. 6 is a flowchart illustrating an example process for updating time interval data throughout a treatment period.

FIG. 7 is a diagram illustrating components of an energy emitting device shown according to the principles of the present disclosure.

FIG. 8A is a diagram illustrating an example of a power sensing unit of the energy emitting device of FIG. 7.

FIG. 8B is another diagram illustrating an example of the power sensing unit of the energy emitting device of FIG. 7.

FIG. 9A is a diagram illustrating dynamic PWM without controlling the drive level.

FIG. 9B is another diagram illustrating dynamic PWM without controlling the drive level.

FIG. 10 is a flowchart illustrating an example process for delivering power using dynamic PWM but without controlling drive level.

FIG. 11A is a diagram illustrating dynamic PWM with control over drive level.

FIG. 11B is another diagram illustrating dynamic PWM with control over drive level.

FIG. 11C is another diagram illustrating dynamic PWM with control over drive level.

FIG. 12 is a flowchart illustrating an example process for dynamic PWM with control over drive level.

FIG. 13 is a diagram illustrating dynamic amplitude modulation.

FIG. 14 is a flowchart illustrating an example process for dynamic amplitude modulation.

### DETAILED DESCRIPTION

The following detailed description of example embodiments refers to the accompanying drawings. The same reference numbers in different drawings may identify the same or similar elements.

In humans at rest, a skin temperature of around 33° C. is experienced as thermoneutral, with lower temperatures felt as cool or cold and higher temperatures felt as warm or hot. In particular, temperatures above about 43° C. evoke acute pain. Classical experiments on porcine and human skin have shown that exposure of the skin to a temperature as low as 44° C. causes cutaneous burn injury when sustained for a period of approximately six hours. Moreover, the rate of injury rises rapidly with increasing temperature, such that for each degree rise in surface temperature the time to produce such injury approximately halves. As such, temperatures above 50° C., burn injuries occur on the seconds timescale and above 60° C. on the sub seconds timescale, requiring rapid heat detection and withdrawal response to avoid serious tissue damage.

The detection of painful thermal stimuli commences when peripheral terminals of nociceptive C fibers (nonmyelinated) and A8 fibers (thinly myelinated) in the skin and mucosae become depolarized in response to noxious temperatures, leading to the initiation of trains of action potentials. These neurons have a characteristic pseudo unipolar morphology, with a single axon that bifurcates into a peripheral branch and a central branch. Via this axon, action potentials that originate in the periphery are transmitted toward the presynaptic terminal, where the detected information is relayed onto second-order sensory neurons and interneurons. The cell bodies of nociceptive and non-nociceptive primary sensory neurons are contained in the dorsal root ganglia (DRG), just lateral to the spinal cord, and in the trigeminal ganglia (TG), located in the middle cranial fossa adjacent to the brain.

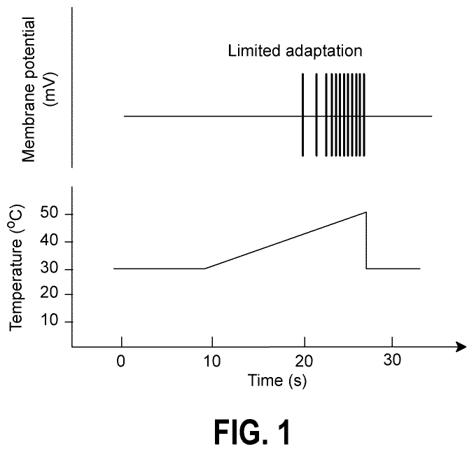

Nociceptive DRG neurons, including those that convey cold and heat pain, have synaptic endings in lamina I, II, and V of the spinal cord dorsal horn. There, glutamate is released onto second-order sensory neurons, which cross the midline and project to the thalamus via a contralateral ascending spinothalamic tract. Signals that travel via the spinothalamic tract are decoded by the thalamus, sensorimotor cortex, insular cortex, and the anterior cingulate, resulting in the perception of an unpleasant sensation localized to a specific region of the body. Action potentials ascending the spinobulbar tract are decoded by the amygdala and hypothalamus to generate a sense of urgency and intensity. Sensory neurons that respond to noxious heat are mainly silent at thermoneutral temperatures but show sustained rapid action potential firing in response to prolonged noxious thermal stimuli. Indeed, thermal stimuli are coded by the action potential firing patterns of different types of temperature-sensitive primary sensory neurons, the cell bodies of which are located in the dorsal root and trigeminal ganglia. Humans experience little or no adaptation to noxious temperatures. Fibers involved in sensing noxious heat are typically activated at temperatures above 43° C., with peak discharge occurring at noxious temperatures (45-53° C.), and show little or no adaptation, as shown in FIG. 1.

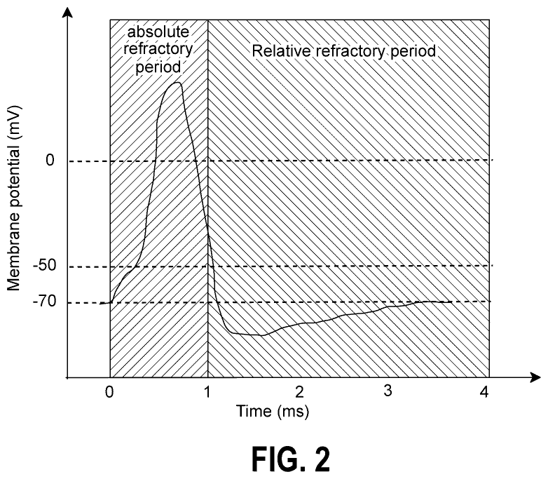

The generation of action potential in neurons is characterized by the all-or-nothing principle. The all-or-nothing principle states that the strength (or amplitude) of the action potential is not dependent upon the strength of the stimulus. If the stimulus is above a certain threshold, an action potential is fired. Essentially, there will either be a full response or there will be no response at all in a neuron to a stimulus. Physiologically, action potential frequencies of up to 200-300 Hertz (Hz) per second are routinely observed. Higher frequencies are also observed, but the maximum frequency is limited by the absolute refractory period (ARP). The ARP refers to the period in which the voltage-gated sodium channels remain inactive, meaning the sodium channels will not open in response to depolarization, as shown in FIG. 2. Because the absolute refractory period is approximately 1 millisecond (ms), there is a limit to the highest possible frequency at which neurons can respond to strong stimuli, which is about 1000 Hz. Therefore, if the noxious heat persists for no longer than 1 ms, by the time the axon is ready to carry another action potential, the action potential may no longer be generated at the nerve endings.

Many energy emitting devices cannot generate a uniform temperature across the intended treatment site or volume. For example, RF electrodes based devices, regardless of monopolar or bipolar, conductively coupled or capacitively coupled, suffer from edge effects, thereby resulting in temperature hotspots. RF devices, or microwave non-contact antenna or antenna array-based devices typically generate non-uniform temperature in the treatment site or volume, due to the anisotropic radiation pattern and constructive or destructive interferences. High intensity focused ultrasound, on the other hand, intentionally focuses the acoustic energy several millimeters underneath the skin surface to form a temperature hotspot. Because the pain sensation is caused by the temperatures above the nociceptor reporting threshold, as well as by the duration for which such noxious temperatures stay above the threshold, it is critical to avoid excessive temperature hotspot build-up, or to make its lifetime shorter than the time needed to form the coded action potential firing pattern.

Therefore, it is important to consider two factors to alleviate noxious heat induced pain: first, to reduce the temperature build-up in hotspot areas as much as possible; second, to reduce or minimize the duration during which such hotspots stay above nociceptor reporting threshold as much as possible. The physiological implication of the former is that the hotspot temperature should not exceed the nociceptor reporting threshold in the first place to avoid firing action potential that could be perceived by central nervous system (CNS) as noxious pain. The physiological implication of the latter is that the duration of the temperature hotspot shouldn't exceed the time needed to form trains of coded action potentials that are interpreted by the CNS as noxious pain. For example, if the noxious pain sensation requires five consecutively fired action potentials, each with a duration of approximately 2 ms, then total train takes about 10 ms to complete; if the hotspot temperature falls below the reporting threshold well before 10 ms time, it may not be able to fire five consecutive action potentials, thus the pain sensation may be alleviated.

Energy emitting devices can implement a number of different techniques for controlling the amount of power or signal intensity in an electronic system. Pulse width modulation (PWM) is a technique that involves rapidly turning a signal on and off at varying durations. Different PWM schemes may be implemented to prevent excessive temperature build-up in the hotspot or prevent the lifetime of the hotspot from being excessively long. Ideally, the energy delivered in the hotspot should be substantially thermally relaxed during PWM low time to avoid substantial accumulated temperature build-up in the hotspot. This thermal relaxation happens by transmitting the heat in the hotspot to adjacent tissue by, for example, thermal conduction, thermal radiation, or thermal convection. Likewise, the PWM frequency should be sufficient so as to prevent PWM high time from being excessively long. This will prevent activation of nociceptors within a single PWM high time. Thermodynamically, thermal relaxation time is related to the size of the hotspot. One way to estimate the thermal relaxation time (measured in seconds) of a hotspot is to determine the thermal relaxation time as being proportional to the square of its size (measured in millimeters). For example, if the hotspot is about 0.1 mm, it will take 0.01 seconds for the hotspot to reach a thermally relaxed state, i.e., to dissipate approximately 63% of the energy received at the hotspot.

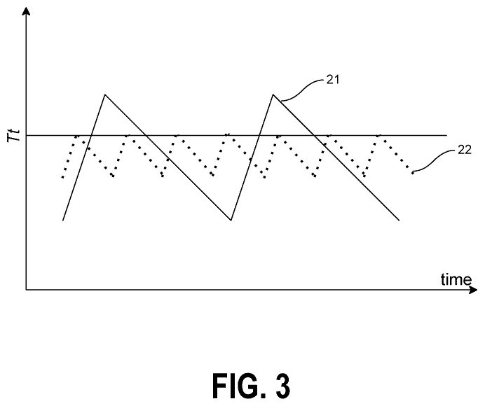

FIG. 3 shows a first temperature profile 21 and a second temperature profile 22. The first temperature profile 21 corresponds to a first PWM of 10 Hz and the second temperature profile corresponds to a second PWM of 100 Hz. The PWM's corresponding to each respective temperature profile share the same exemplary power duty (or otherwise known as duty cycle) of 40%, thus all other conditions equal, the average energy delivery in the hotspot will be the same for each PWM. However, due to the 40 ms PWM high-time associated with the first PWM, the temperature in the hotspot corresponding to the first PWM experiences a much greater increase compared to the temperature in the hotspot corresponding to the second PWM, where the PWM high-time is only 4 ms. The first temperature profile 21 may exceed the nociceptor activation threshold (Tt), which may result in the nociceptor firing action potentials leading to a noxious pain sensation. The second temperature profile 22 may stay below the threshold Tt, meaning no nociceptor action potential will be fired. Effectively, by reducing the PWM period time (or increasing PWM frequency), the temperature fluctuation in the hotspot may be substantially flattened, thereby avoiding activating the nociceptors.

For a safe and efficacious energy emitting device treatment, it is essential to deliver power to the tissue substantially equal to an amount of power needed for a given treatment procedure. However, as mentioned elsewhere herein, load impedance changes constantly and is dependent on a variety of factors, including electrical properties of the biological tissue, patient movement, tissue perfusion, the coupling medium, contact pressure, and/or the like. This results in the instantaneous received power constantly fluctuating even when the drive level of the energy emitting device remains the same. Consequently, delivering the instantaneous received power equal to or substantially equal to the requisite amount of electromagnetic energy needed for a given treatment procedure poses significant engineering challenges.

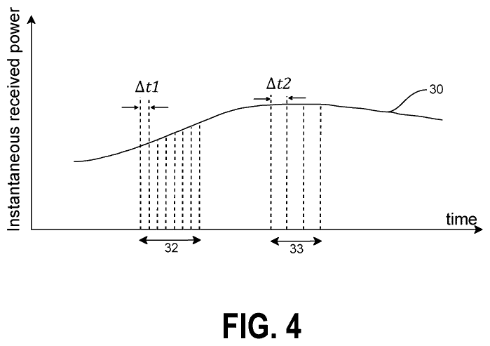

FIG. 4 shows is a diagram illustrating an instantaneous received power curve 30 measured relative to time. As can be seen in FIG. 4, the instantaneous received power curve 30 may be represented over a set of time intervals. Within each time interval Δt, a variation between instantaneous received power values is less than a threshold tolerance level. The slope of the instantaneous received power curve 30 may change depending on the nature of the treatment and/or the technique implemented by the operator of the energy emitting device. The greater the slope, the smaller the time interval Δt that is needed to ensure that variations between instantaneous received power values remain below the threshold tolerance level. The graph in FIG. 4 shows a region 32 that corresponds to time interval Δt1 and a region 33 that corresponds to time interval Δt2. As can be seen, in region 32, the slope of the curve 30 is steeper than in region 33. Consequently, the time interval Δt1, which corresponds to region 32 (i.e., where curve 30 has a steeper slope), has to be shorter than time interval Δt2, which corresponds to region 33 (i.e., where curve 30 has a flatter slope).

In some embodiments, a time interval Δt may be a time between 1 nanosecond (ns) and 10 seconds(s). In some embodiments, the time interval Δt may be a time between 1 microsecond (us) and 1 s. In some embodiments, the time interval Δt may be a time between 10 us and 100 ms. In some embodiments, the time interval Δt may be a time between 100 us and 10 ms.

As used herein, the term “instantaneous received power” refers to the average power at the input of the electromagnetic energy delivery unit 43 over one period of the electromagnetic energy, which can be expressed as:

$$P = \frac{1}{T} {\int }_{t}^{t + T} V ( t ) \cdot I ( t ) d ( t ) ,$$

where P is the instantaneous received power, t is time, and T is the period of the electromagnetic energy, V(t) and I(t) are the voltage and current at the input of the electromagnetic energy delivery unit 43, respectively.

One or more embodiments described herein refer to a treatment energy delivery value. The treatment energy delivery value identifies an amount of electromagnetic energy that needs to be delivered over a configured period of time (e.g., such as a pulse width modulation (PWM) period, a time interval, etc.). The treatment energy delivery value may be represented using energy measured in Joules (J) or power measured in watts (W). When the treatment energy delivery value is represented in Joules, it can be readily converted to power in watts within the time interval Δt as commonly understood by one of ordinary skill in the art. Similarly, when the treatment energy delivery value is represented in watts, it can be readily converted to Joules, as would be understood by one of ordinary skill in the art.

In some embodiments, the treatment energy delivery value may be configured based on the type of treatment being performed. That is to say, different types of treatment may require different power delivery values. Additionally, or alternatively, the treatment energy delivery value may be configured based on the expertise of the operator or physician performing the treatment procedure and/or any other factors known in the art. In some embodiments, the treatment energy delivery value may change one or more times throughout a treatment procedure. For example, a treatment plan may require a first treatment energy delivery value to be delivered during a first interval of the treatment procedure and may require a second treatment energy delivery value to be delivered during a second interval of the treatment procedure.

While one or more embodiments described herein use the term “load impedance,” it is to be understood that this is provided by way of example. In practice, a number of other electromagnetic energy delivery parameters may be utilized, including voltage standing wave ratio (VSWR), return loss, reflection coefficient, and/or the like. These parameters are mathematically related to load impedance as would be understood by one of ordinary skill in the art.

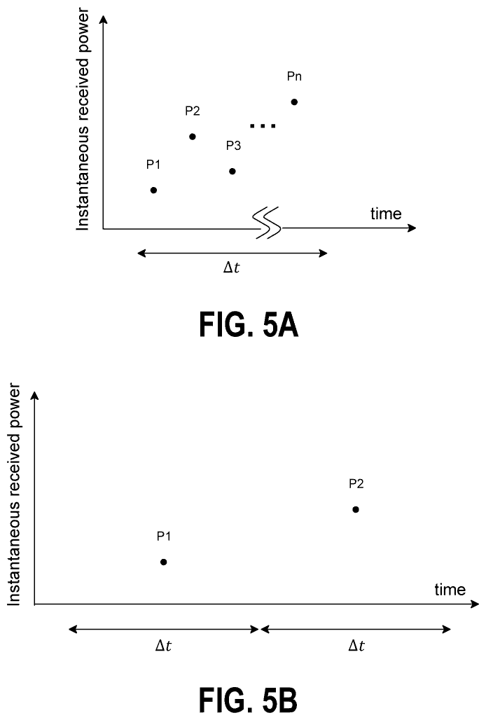

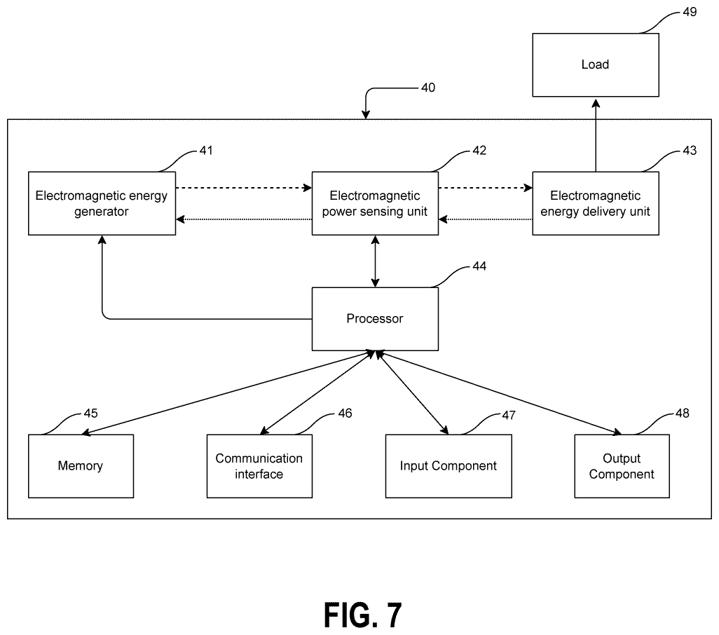

FIGS. 5A and 5B are diagrams illustrating graphical representations of an energy emitting device determining one or more instantaneous received power values over one or more time intervals. An energy emitting device includes a generator, an electromagnetic sensing unit, an electromagnetic energy delivery unit, a processor, and one or more other components described in connection with FIG. 7.

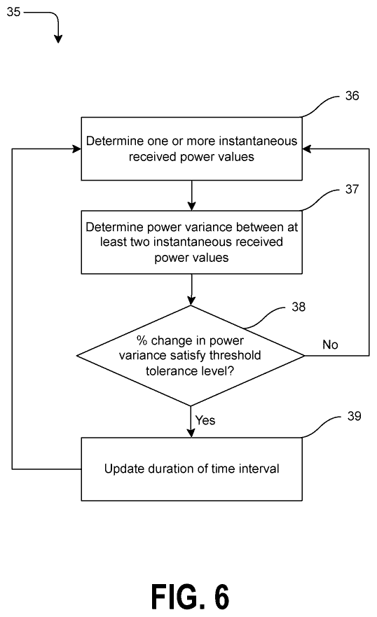

FIG. 5A shows the energy emitting device determining multiple instantaneous received power values during a time interval Δt. For example, the energy emitting device (e.g., using the electromagnetic sensing unit) may determine a set of instantaneous received power values (shown as P1, P2, P3, . . . , Pn) during the time interval Δt. A description of how an instantaneous received power value is determined is provided in connection with FIG. 6 below.

FIG. 5B shows the energy emitting device determining a single instantaneous received power value during each respective time interval Δt. For example, the energy emitting device (e.g., using the electromagnetic sensing unit) may determine a first instantaneous received power value (shown as P1) during a first time interval and may determine a second instantaneous received power value (shown as P2) during a second time interval. A description of how an instantaneous received power value is determined is provided in connection with FIG. 6 below.

FIG. 6 is a flowchart illustrating an example process 35 for updating time interval data throughout a treatment period. For example, the energy emitting device may update time interval data throughout a duration of a treatment period, where the treatment period represents a total time during which to deliver electromagnetic energy as part of a treatment procedure.

In some embodiments, the time interval data may include data identifying a single time interval that is updated throughout a treatment procedure. In some embodiments, the time interval data may include data identifying a set of time intervals (sometimes referred to as discretized time intervals). The set of time intervals may occur in succession such that the set of time intervals extend throughout the duration of the treatment period. In this case, the energy emitting device may update one or more of the time intervals by updating any subsequent time intervals that have yet to occur. For example, assume the set of time intervals include sixty time intervals of 1 millisecond (ms) each (e.g., which occur over a 60 ms treatment period). Further assume that after the first 1 ms time interval, the energy emitting device determines to update the set of time intervals to 0.5 ms. In this example, the energy emitting device may update the time interval data such that data identifying the second, third, . . . , sixtieth time interval is updated to a value of a 0.5 ms. It is noted that this increases the total number of time intervals during the treatment period. For example, if there are five time intervals of 1 ms, and after the first time interval of 1 ms, the duration is decreased to 0.5 ms, then there will now be eight 0.5 ms time intervals occurring over the remaining 4 ms of the treatment period.

In some embodiments, the energy emitting device may be configured with time interval data identifying an initial set of time intervals. The initial set of time intervals may extend throughout the duration of the treatment period. For example, if a treatment plan specifies that electromagnetic energy is to be delivered throughout a 60 milliseconds (ms) treatment period, the energy emitting device may be configured with time interval data identifying sixty 1 ms time intervals. As will be described below, the duration of these intervals may be updated throughout the treatment period.

The energy emitting device may determine one or more instantaneous received power values during each respective time interval (step 36). In some embodiments, the energy emitting device (e.g., using the electromagnetic power sensing unit) may determine the one or more instantaneous received power values based on measured instantaneous power levels. The measured instantaneous power levels may include measured instantaneous forward power levels and measured instantaneous reflected power levels. In this case, the electromagnetic power sensing unit may measure an instantaneous forward power level and an instantaneous reflected power level and data indicative of these measurements may be provided to the processor of the energy emitting device. Next, the energy emitting device (e.g., using the processor) may determine the instantaneous received power value based on the measured instantaneous power levels. For example, the energy emitting device may determine an instantaneous received power value by subtracting data identifying an instantaneous reflected power level from data identifying an instantaneous forward power level.

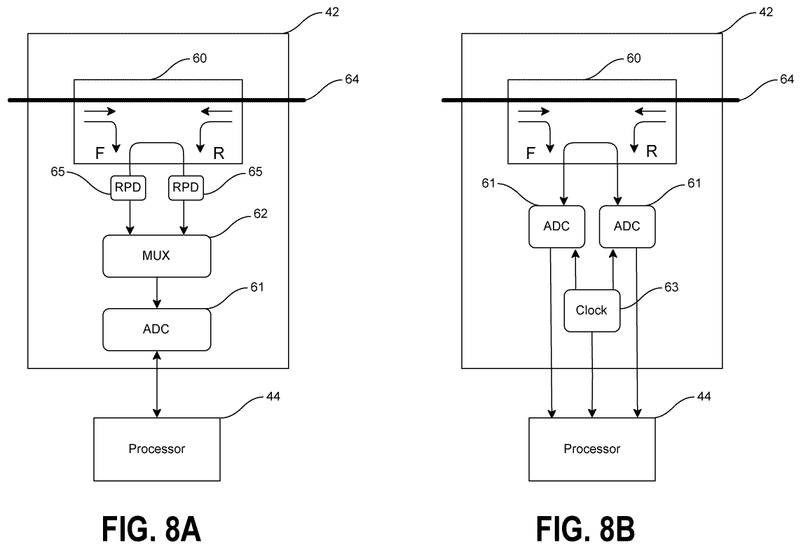

While one or more embodiments describe instantaneous received power values as being determined based on instantaneous power levels (forward and reflected), it is to be understood that this is provided by way of example. In practice, the instantaneous received power values may be determined in any other manner known in the art. For example, the instantaneous received power values may be determined using voltage and current measurements, as is described in connection with FIGS. 8A and 8B.

The energy emitting device may determine a change in power variance between at least two instantaneous received power values (step 37). For example, the energy emitting device (e.g., using the processor) may determine a power variance using Equation 1 and Equation 2 below:

$$PV = {P}_{max} - {P}_{min} \qquad ( 1 )$$

$$\% Change in PV = \frac{( PV )}{P min} \qquad ( 2 )$$

In Equations 1 and 2, PV represents a power variance value, Pₘₐₓ represents a maximum instantaneous received power value, and Pₘᵢₙ represents a minimum instantaneous received power value. To compute the power variance, the energy emitting device may subtract a minimum instantaneous received power value (Pₘᵢₙ) from a maximum instantaneous received power value (Pₘₐₓ). To compute the change in power variance, the energy emitting device may divide the power variance by the minimum instantaneous received power value (Pₘᵢₙ). To provide an example, assume the following instantaneous received power values: 10 watts (W) at time t1, 12 W at time t2, 15 W at time t3, and 18 W at time t4. In this example, the maximum instantaneous received power value is 18 W and the minimum instantaneous received power value is 10 W. Using Equation 1 above, the energy emitting device determines that the power variance (PV) is 8 W and may determine that the change in power variance is 0.8 or 80%

$$( e . g . , \frac{8 W}{10 W} ) .$$

In some embodiments, the energy emitting device may determine a power variance between two or more instantaneous received power values in the same time interval. In some embodiments, only one instantaneous received power value is determined during each time interval. In this case, the energy emitting device may determine a power variance between an instantaneous received power value of a current time interval and an instantaneous received power value of a previous time interval.

The energy emitting device may determine whether a change in power variance satisfies a threshold tolerance level (step 38). For example, the energy emitting device may be configured with data identifying one or more threshold tolerance levels. The one or more threshold tolerance levels may include an upper bound threshold tolerance level and/or a lower bound threshold tolerance level. The upper bound threshold tolerance may represent a maximum permissible change in the power variance. For example, if the power variance is above the upper bound threshold tolerance level, the energy emitting device may need to decrease the time interval so that more frequent computations can be performed (e.g., in order to more effectively manage the variations in power delivery). The lower bound threshold tolerance may represent a power variance that is so low that the energy emitting device is operating inefficiently. For example, if the power variance is below the lower bound threshold tolerance, then the energy emitting device may be performing computations more frequently than is needed.

In some embodiments, the upper bound threshold tolerance level may be less than 200%. In some embodiments, the upper bound threshold tolerance level may be less than 100%. In some embodiments, the upper bound threshold tolerance level may be less than 20%. In some embodiments, the upper bound threshold tolerance level may be less than 5%.

If the change in power variance satisfies the threshold tolerance level, the energy emitting device may update the duration of the time interval (step 39). For example, if the change in power variance satisfies (e.g., exceeds) the upper bound threshold tolerance level, the energy emitting device may decrease the duration of the time interval. If the change in power variance satisfies (e.g., exceeds) the lower bound threshold tolerance level, the energy emitting device may increase the duration of the time interval.

In some embodiments, the energy emitting device may determine a degree to which to update the duration of the time interval. For example, the energy emitting device (e.g., using the processor) may execute a proportional-integral-derivative (PID) control technique or a similar type of technique to determine the degree to which to update the duration of the time interval.

If the change in power variance does not satisfy the threshold tolerance level, the energy emitting device may continue determining one or more instantaneous received power values (step 36).

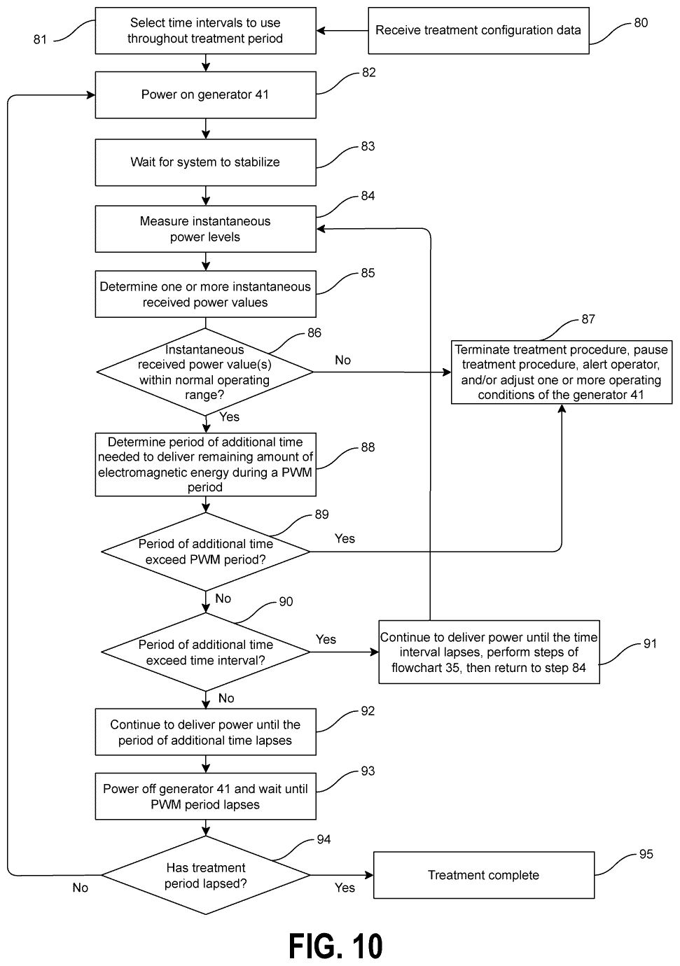

As will be shown in FIGS. 10, 12, and 14, the energy emitting device can perform a number of different computations to manage power delivery throughout the treatment period. By managing or updating the duration of time intervals throughout the treatment period, the energy emitting device is able to increase or decrease the frequency at which these computations are performed, thereby efficiently and effectively managing the power delivery throughout the treatment period. Furthermore, by managing or updating the duration of time intervals, the energy emitting device is able to ensure to variations in instantaneous received power do not vary to a level that will cause the output power to trigger pain receptors in the patient receiving treatment. Similar ranges may be utilized in connection with the lower bound threshold tolerance level.

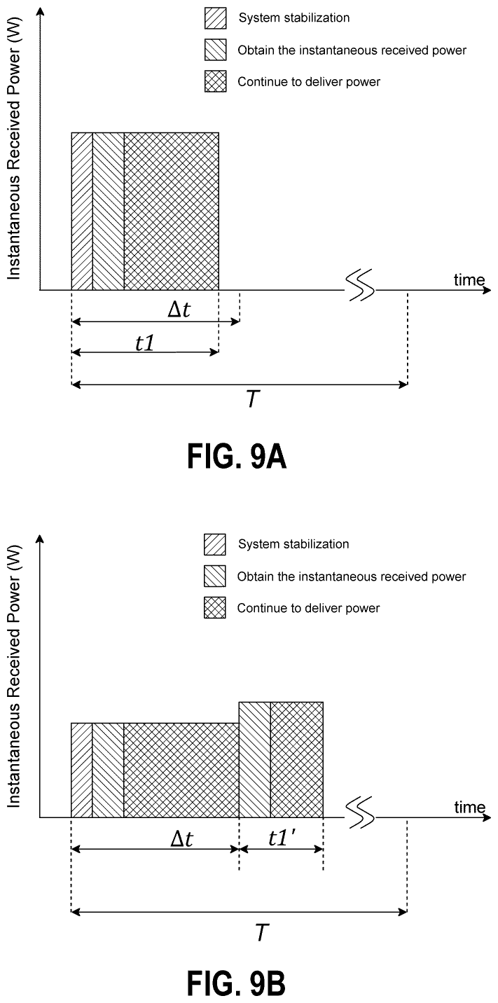

One or more embodiments described in connection with FIGS. 9A and 9B, 10, 11A-11C, and 12-14 may utilize this process for updating the duration of the time interval.

FIG. 7 is a diagram illustrating components of an energy emitting device 40 shown according to the principles of the present disclosure. Energy emitting device 40 includes an electromagnetic energy generator 41, a power sensing unit 42, an electromagnetic energy delivery unit 43, a processor 44, a memory 45, a communication interface 46, an input component 47, and an output component 48. A bus or a related piece of hardware may be used to permit communication among multiple components of the energy emitting device 40.

The electromagnetic energy generator 41 generates electromagnetic energy in the frequency range from ˜30 kHz to ˜30 GHz, more preferably in the range from 300 kHz to 3 GHz.

The power sensing unit 42 measures the instantaneous received power. The power sensing unit 42 may be an electromagnetic power sensing unit. Any number of different sensing units may be implemented to fit the needs of a given treatment procedure.

The electromagnetic energy delivery unit 43 converts the electromagnetic energy generated by the electromagnetic energy generator 41 to various energy forms that are received by a load 49. The energy forms produced by the electromagnetic energy delivery unit 43 include RF current, RF electromagnetic field, electromagnetic radiation, ultrasound wave, laser, and/or any combination thereof. The load 49 in the present invention, for simplicity and clarity, primarily refers to the human tissue to be treated. The load impedance in the present invention refers to the electrical impedance at the input of the electromagnetic energy delivery unit 43. The electrical impedance of the human tissue, the coupling mechanism and the coupling material, and the inherent impedance of the electromagnetic energy delivery device 43 itself, may all contribute to the load impedance.

Often times the electromagnetic energy delivery unit 43 is called an applicator, therefore electromagnetic energy delivery unit and applicator are interchangeable in the present invention. The electromagnetic energy delivery unit 43 may comprise electrodes that are in direct contact with the tissue to be treated to deliver RF current. The electromagnetic energy delivery unit 43 may comprise electrodes that are not in direct contact with the tissue to be treated to deliver RF electromagnetic field; the electrodes and the tissue are often separated by a thin dielectric material (or air) that functions to block electrical current but allow the field energy to couple into the tissue. The electromagnetic energy delivery unit 43 may comprise ultrasonic transducers to deliver acoustic pressure waves to the tissue needs to be treated; coupling gel may be placed in between the ultrasonic transducer and the tissue to assist in energy coupling. The electromagnetic energy delivery unit 43 may comprise antenna structures to deliver electromagnetic radiation in the form of microwaves through antenna structures to the tissue to be treated, the antenna structures may include monopole antenna, dipole antenna, coaxial single slot antenna, coaxial multiple slot antenna, waveguide antenna, horn antenna, patch antenna, patch trace antenna, Vivaldi antenna; the antenna structures may or may not be placed in direct contact with the tissue to be treated. Without wishing to be bound by theory, the electromagnetic energy delivery unit 43 may convert electromagnetic energy to energy forms including but not limited to RF current, RF electromagnetic field, electromagnetic radiation, ultrasound wave, and laser, simultaneously or in series during one session of a treatment.

Processor 44 is implemented in hardware, firmware, and/or a combination of hardware and software. Processor 44 includes a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), and/or another type of processing component. In some embodiments, processor 44 includes one or more processors capable of being programmed to perform a function.

The processor 44 is in direct electrical communication with the electromagnetic energy generator 41. The processor 44 may adjust the operating conditions of the electromagnetic energy generator 41. Adjusting the operating conditions may include changing or toggling a state of the generator 41 (e.g., between an on state and an off state), and/or may include increasing or decreasing the drive level on the generator 41. Typically, increasing the drive level on the electromagnetic energy generator 41 increases the power output of the electromagnetic energy generator 41, and vice versa.

The processor 44 is in communication with the power sensing unit 42, to direct the power sensing unit 42 to measure the instantaneous received power at least once within each discretized time interval Δt. According to various embodiments of the present invention, obtaining the instantaneous received power comprises measuring the instantaneous forward and reflected power, respectively. The instantaneous received power may be calculated by subtracting the instantaneous reflected power (or abbreviated to reflected power) from the instantaneous forward power (or abbreviated to forward power). The instantaneous forward power refers to the power that travels from the electromagnetic energy generator 41 to the electromagnetic energy delivery unit 43, as shown by the long-dashed arrow in FIG. 6. The instantaneous reflected power refers to the power that travels from the electromagnetic energy delivery unit 43 to the electromagnetic energy generator 41, as shown by the short-dashed arrow in FIG. 6. It is important to note that, similar to the definition of instantaneous received power in the present invention, the instantaneous forward and reflected power are defined over one period of the electromagnetic energy, however, due to hardware and sampling speed limitation of the power sensing unit 42, measuring the instantaneous forward and reflected power, thus the instantaneous received power, typically may take more than one period of the electromagnetic energy, as commonly known by one with ordinary skills in the art.

Memory 45 includes a random-access memory (RAM), a read only memory (ROM), and/or another type of dynamic or static storage device (e.g., a flash memory, a magnetic memory, and/or an optical memory) that stores information and/or instructions for use by processor 44.

The processor 44 may use the communication interface 46 to communicate with one or more external devices. Communication interface 46 includes a transceiver-like component (e.g., a transceiver and/or a separate receiver and transmitter) that enables energy emitting device 40 to communicate with other devices, such as via a wired connection, a wireless connection, or a combination of wired and wireless connections. Communication interface 46 may permit energy emitting device 40 to receive information from another device and/or provide information to another device. For example, communication interface 46 may include an Ethernet interface, an optical interface, a coaxial interface, an infrared interface, a radio frequency (RF) interface, a universal serial bus (USB) interface, a Wi-Fi interface, a Bluetooth interface, a cellular network interface, and/or the like.

The processor 44 may be in communication with input component 47 to receive inputs from a device operator such as a physician or technician. Input component 47 includes a component that permits energy emitting device 40 to receive information, such as via user input (e.g., a touch screen display, a keyboard, a keypad, a mouse, a button, a switch, and/or a microphone). Output component 48 includes a component that provides output information from energy emitting device 40 (e.g., a display, a speaker, and/or one or more light-emitting diodes (LEDs), etc.).

As shown in FIGS. 8A and 8B, the power sensing unit 42 may decouple the instantaneous forward power and reflected power from the main power line 64. The main power line 64 may be coaxial cable or waveguide, among other forms. The power sensing unit 42 may comprise two separate directional couplers (not shown in FIG. 6) with each sensing the instantaneous forward and reflected power, respectively. Alternatively, the power sensing unit 42 may comprise one bi-directional coupler 60. Signal coming out from port F is directly related to the instantaneous forward power traveling in the main power line 64, signal coming out from port R is directly related to the instantaneous reflected power traveling also in the main power line 64. The signals may each pass through a RF peak detector 65 (RPD) which converts an alternating current (AC) signal into a direct current (DC) signal. The amplitude of the DC signal is proportionally related to the instantaneous forward or reflected power traveling in the main power line 64. The DC signals representing the instantaneous forward and reflected power traveling in the main power line 64 may be optionally connected to a multiplexer 62, which is then connected to one or more analog to digital converters (ADCs) 61. An ADC may be in electrical communication with the processor 44. The processor 44 directs the ADC to sample, at least once, the DC signals within each discretized time interval Δt. The processor 44 then calculates, at least once, the instantaneous received power by subtracting the instantaneous reflected power from the instantaneous forward power.

In some embodiments, the AC signals from port F and port R maybe in direct electrical communication with two ADCs 61 without passing through RF peak detectors. The two ADCs may rely on a clock unit 63 to provide trigger to initiate sampling. This clock unit 63 is configured to direct the two ADCs to measure the instantaneous forward and reflected power signals either synchronously or non-synchronously, within each discretized time interval Δt. Each measurement on the instantaneous forward and reflected power signals may require sampling the respective signal more than once.

In some embodiments, the ADCs employ baseband sampling, i.e., the sampling frequency is greater than twice the signal frequency of interest. In some embodiments, the ADCs employ under sampling. According to Nyquist theorem of sampling, under sampling only requires the sampling frequency to be greater than twice the Nyquist's signal bandwidth, essentially allowing a much lower ADC sampling frequency to be used to sample a high frequency signal. The processor 44 may be directed by the clock unit 63 to retrieve the digitized instantaneous forward and reflected power signals from the two ADCs and store them in its memory for further processing. The digitized instantaneous forward and reflected power signals may each be mathematically processed in the processor 44 to calculate their amplitude.

One form of mathematical processing involves Fourier Transformation. After performing Fourier Transformation to each sampled signal, their amplitude at the frequency of interest can be calculated. Then the instantaneous forward and reflected power can be back calculated depending on the attenuation provided by the directional or bi-directional coupler 60. Another advantage of using Fourier Transformation is it can reveal the amplitude information of other harmonics that are missing in the peak/peak to peak detector method. The processor 44 then calculates the instantaneous received power by subtracting all the instantaneous forward power from all the instantaneous reflected power (i.e., power of each harmonic). In alternative embodiments, the two ADCs may be controlled directly by the processor 44 to initiate sampling and send to the processor 44 the digitized forward and reflected instantaneous power signals.

In some embodiments, obtaining the instantaneous received power within each discretized time interval Δt comprises measuring the voltage V(t), and the current I(t) at the input of the electromagnetic energy delivery unit 43. The instantaneous received power may then be determined by the processor 44 by integrating the product of the voltage V(t) and current I(t) over one period of the electromagnetic energy. Baseband sampling and/or under sampling may be employed to measure the voltage V(t) and current I(t), the processor 44 may then utilize Fourier Transformation to process the two waveforms. According to some alternative embodiments of the present invention, obtaining the instantaneous received power may comprise measuring the RMS values of the voltage V(t), and current I(t), and the phase angle θ between the voltage V(t) and current I(t). The instantaneous received power may then be readily calculated by the processor 44 as the product of the RMS values of V(t) and I(t), and cos θ, which may be expressed as V_{RMS}×I_{RMS}×cos θ, where V_{RMS} is the RMS value of the voltage V(t), and I_{RMS} is the RMS value of the current I(t).

It should be appreciated that the foregoing methods for obtaining the instantaneous received power within each discretized time interval Δt do not intend to be exhaustive and may be modified without departing from the spirit and scope of the present invention.

In some embodiments, the processor 44 may select a pre-determined time interval Δt value. For example, the processor 44 may select a pre-determined time interval Δt value for a specific treatment plan. In this case, the processor 44 may store pre-determined time interval Δt values for different treatment plans and may select a value based on the treatment plan being performed. In this case, time interval Δt values may be configured based on prior knowledge of the rate of change of the instantaneous received power for the treatment plan. Additionally, or alternatively, a time interval Δt value may be selected based on device information relating to the energy emitting device and/or based on any other information available to the processor 44. In some embodiments, the time interval Δt value may be selected based on user input.

In some embodiments, the processor 44 may constantly obtain the instantaneous received power, analyze the rate of change, and dynamically assign an appropriate time interval Δt (e.g., which may vary over time). In some embodiments, when the processor 44 detects the rate of change of the instantaneous received power is too high such that the current selected time interval Δt may result in a variation in the instantaneous received power exceeding the tolerance, the processor 44 may reduce the time interval Δt. Likewise, when the processor 44 detects the rate of change of the instantaneous received power is low such that increasing the current time interval Δt may result in a variation in the instantaneous received power that is still within the tolerance, the processor 44 may increase the time interval Δt.

In some embodiments, the processor 44 may monitor and analyze other electromagnetic energy delivery parameters in addition to the instantaneous received power, including VSWR, return loss, reflection coefficient, and/or the like. It should be noted that the instantaneous received power, instantaneous forward power, instantaneous reflected power, and other electromagnetic energy delivery parameters are all mathematically related as commonly understood by one with ordinary skill in the art. These parameters maybe indicative of many aspects of the treatment quality as well as a condition of the energy emitting device. In some embodiments, abnormal values of these parameters may indicate that there is poor coupling between the electromagnetic energy delivery unit 43 and the load 49. These poor coupling may result from insufficient amount of coupling material, unwanted trapped air-pocket, partial tissue contact, and/or the like. In these cases, poor coupling needs to be corrected to ensure safe and efficacious treatment. In some embodiments, abnormal values of these parameters may indicate that the electromagnetic energy delivery unit 43 may have components degradation, contamination, and even malfunction that may compromise treatment efficacy and/or safety. In these cases, the electromagnetic energy delivery unit 43 may need to be serviced.

In some embodiments, the processor 44 may receive data indicative of the measured instantaneous forward and reflected power. In this case, the processor 44 may determine the instantaneous received power, VSWR, return loss, and reflection coefficient within the time interval Δt. The processor 44 may calculate the moving average of the above-mentioned parameters and flag an abnormal condition when a sudden deviation of these parameters from their corresponding moving average exceeds a threshold percentage. In some embodiments, the threshold percentage may be more than 1%. In some embodiments, the threshold percentage may be more than 5%. In a preferred embodiment, the threshold percentage may be more than 10%. The processor 44 may remove the abnormal condition and resume normal treatment operation when the deviation of these parameters return to a value that does not exceed the threshold percentage of their corresponding moving average immediately before the abnormal condition is flagged.

In some embodiments, the processor 44 may implement a predetermined threshold value for the instantaneous forward and reflected power, the calculated instantaneous received power, VSWR, return loss, and reflection coefficient, etc. Once the measured or calculated values cross the threshold, the processor 44 may flag an abnormal condition, and when these values return to within the normal range, the processor 44 may remove the abnormal condition and resume normal treatment operation. The processor 44 may perform one or more actions based on flagging the abnormal conditions. The one or more actions may include shutting down the treatment completely, pausing the treatment, alerting the physician or system operator to take appropriate action, decreasing the power duty to less than configured amount, and/or the like. The configured amount may, for example, be a power value less than 40%, 20%, 10%, 5%, etc. The configured amount may be less than 5% in a preferred embodiment. The power duty refers to power delivery active time divided by the sum of power delivery active time and power delivery inactive time, e.g., in PWM mode, the power duty is the PWM high-time divided by the sum of PWM high-time and PWM low-time for each PWM period.

In some embodiments, the processor 44 may be configured to perform one or more hysteresis algorithms in flagging and removing of an abnormal condition, to increase treatment stability. In one exemplary embodiment, the processor 44 may only remove the abnormal condition when the newly obtained instantaneous received power falls below 90 W, if it previously recorded an abnormal condition when the obtained instantaneous received power exceeded a threshold of 100 W. These abnormal condition monitoring is essential to ensure treatment comfort, treatment safety, and treatment efficacy. Without wishing to be bound by theory, the determination of abnormal values for these parameters may involve other mathematical processes that one with ordinary skills in the art is familiar with.

FIGS. 10, 12, and 14 illustrate processes that can be performed by the energy emitting device 40 to deliver a treatment energy delivery value to biological tissue during a treatment period. The treatment energy delivery value may refer to an amount of electromagnetic energy delivered over a time period (e.g., a PWM period, a time interval, etc.). Each of the processes shown in FIGS. 10, 12, and 14 deliver an amount of electromagnetic energy identified by the treatment energy delivery value by adjusting one or more operating conditions of the generator 41 of the energy emitting device 40. For example, the process shown in FIG. 10 uses dynamic pulse width modulation (PWM) without drive level control to deliver the amount of electromagnetic energy identified by the treatment energy delivery value. In this case, adjusting one or more operating conditions includes adjusting or toggling the generator between an “on” state and an “off” state (thereby varying a width or duration of electrical pulses).

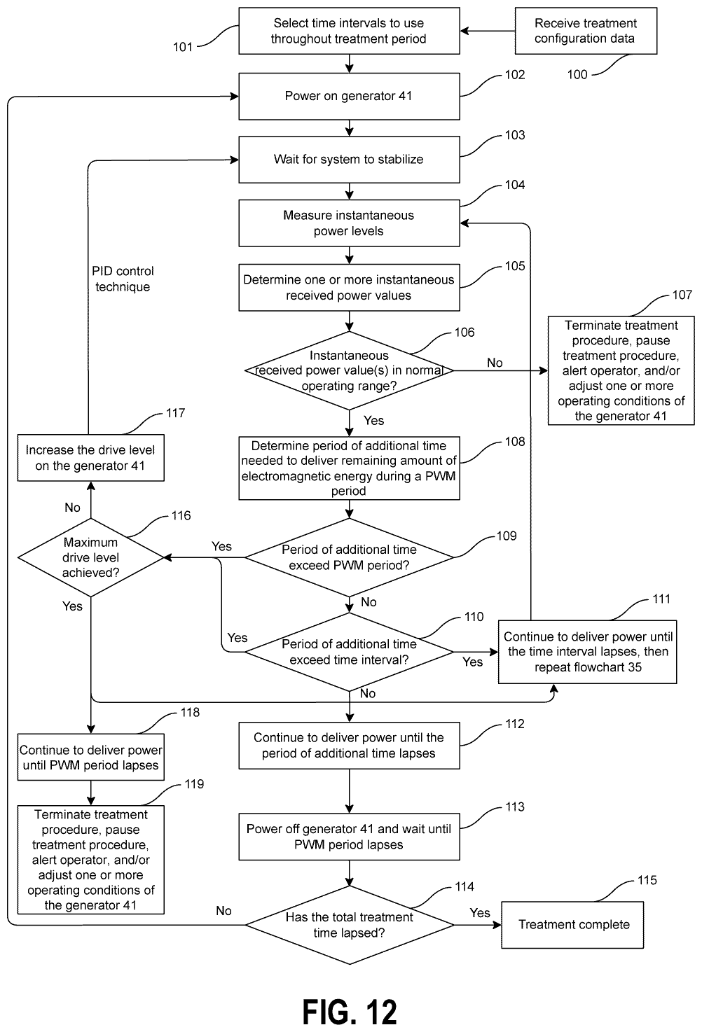

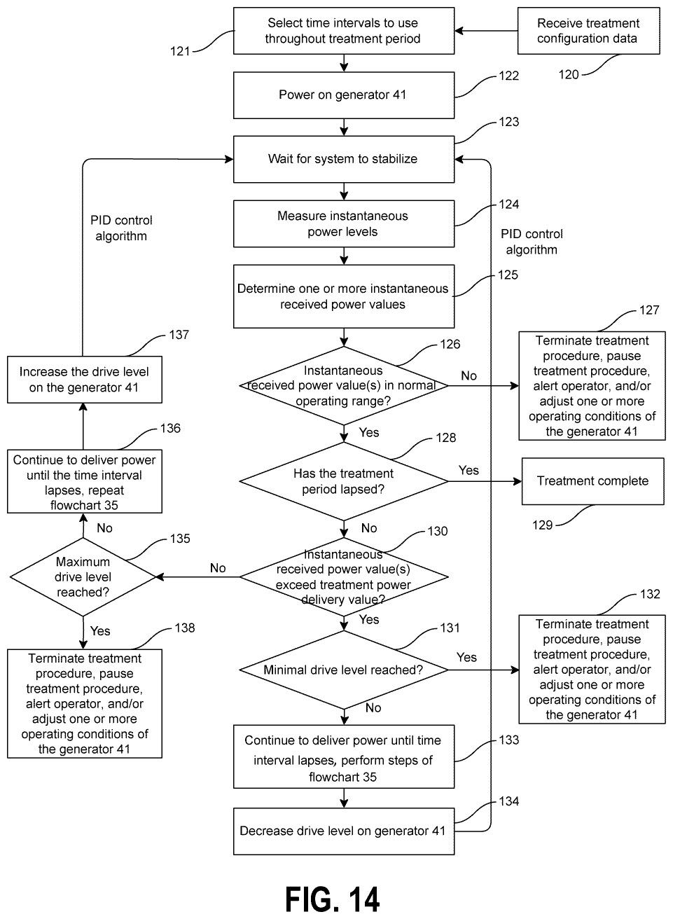

To provide another example, the process shown in FIG. 12 uses dynamic PWM with drive level control to deliver an amount of electromagnetic energy identified by the treatment energy delivery value. In this case, adjusting one or more operating conditions includes adjusting an amount of power delivered to the biological tissue throughout the treatment period. To provide another example, the process shown in FIG. 14 uses dynamic amplitude modulation to deliver the target amount of electromagnetic energy. In this case, adjusting the one or more operating conditions includes one or both of: adjusting or toggling the generator between an “on” state and an “off” state, and adjusting an amount of power delivered to the biological tissue throughout the treatment period.

Now referring to FIGS. 9A, 9B and 10, the energy emitting device 40 may use dynamic PWM without drive level control to deliver electromagnetic energy to biological tissue of a patient. Dynamic PWM involves varying a width or a duration of electrical pulses during the treatment procedure. To control the electromagnetic energy delivered to the biological tissue (e.g., without using drive level control), the energy emitting device 40 may adjust or toggle a state of the generator 41 (e.g., between an on state and an off state), thereby varying the power duty.

The electromagnetic energy may be delivered as part of a treatment procedure. The duration of the treatment procedure, i.e., the total time during which electromagnetic energy is provided to the biological tissue, is referred to as a treatment period.

FIG. 10 illustrates an example process for using dynamic PWM without drive control to deliver electromagnetic energy to biological tissue. The energy emitting device 40 may receive treatment configuration data (step 80). For example, the energy emitting device 40 may receive treatment configuration data for a specific treatment procedure. The treatment configuration data may be input by an operator or administrative user (e.g., via a user interface). The treatment configuration data may include PWM period data, treatment energy delivery data, threshold tolerance level data, initial time interval data, and/or the like.

The PWM period data may include values identifying a set of PWM periods. The set of PWM periods may extend over the duration of the treatment period. A PWM period T represents a duration in which a signal completes an on-and-off cycle.

In some embodiments, the treatment energy delivery data may include a treatment energy delivery value that identifies an expected amount of energy to deliver to the biological tissue over a first PWM period T. In some embodiments, the treatment energy delivery data may include a treatment energy delivery value that identifies an expected amount of energy to deliver to the biological tissue over a first time interval Δt. This embodiment may be used in connection with FIG. 14 (e.g., where PWM periods are not utilized). In the present invention, the treatment energy delivery value may refer to energy in Joules or power in Watts. When the treatment energy delivery value is in Joules, it can be readily converted to power in Watts within the PWM period T or discretized time interval Δt as commonly understood by one of ordinary skills in the art. In some embodiments, the treatment energy delivery value may vary depending on the type of treatment being performed, power delivery capabilities of the energy emitting device 40, etc.

The threshold tolerance data may include a value identifying an upper bound threshold tolerance level and/or a value identifying a lower bound threshold tolerance level. In some embodiments, the initial time interval data may include values identifying durations of a set of time intervals. The time intervals may extend over the duration of the treatment period. In some embodiments, the initial time interval data may include a value identifying a duration of a single time interval Δt. The time interval Δt may be repeated over the duration of the treatment period.

Throughout the description of FIG. 10, an illustrative example will be provided using numerical values. In this example, the energy emitting device 40 may be configured with the following treatment configuration data: a PWM period T value of 10 ms, a treatment energy delivery value of 35 W, a value identifying an upper threshold tolerance level of 0.25 (25%), a value identifying a lower threshold tolerance level of 0.05 (5%), and a set of time interval values where the initial time interval duration is set to 1 ms.

In some embodiments, the energy emitting device 40 may store the treatment configuration data in memory.

Next, the energy emitting device 40 may select a set of time intervals to use throughout the treatment period (step 81). For example, the energy emitting device 40 (e.g., using the processor 44) may select the configured initial time intervals as the set of time intervals to use throughout the treatment period. The duration of these time intervals drives the frequency at which the energy emitting device 40 performs one or more of the steps described below. For example, if the duration of each time interval Δt is configured to 1 ms, then the energy emitting device 40 may perform one or more of the steps of FIG. 10 at least once during each 1 ms time interval. Comparatively, if the time interval Δt is 0.5 ms, then the energy emitting device 40 may perform one or more of the steps of FIG. 10 at least once during each time interval Δt of 0.5 ms (i.e., more frequently relative to the 1 ms example).

In some embodiments, the energy emitting device 40 may determine to update a duration of one or more of the time intervals. For example, the energy emitting device 40 may determine to update the duration of one or more time intervals as described in connection with FIG. 6. In some embodiments, the energy emitting device 40 may determine a degree to which the duration of a time interval Δt is to be updated. For example, the energy emitting device 40 may execute a proportional-integral-derivative (PID) control technique or a similar type of technique to determine the degree to which to update the duration of the time interval Δt.

Next, the energy emitting device 40 may turn on the generator 41 (step 82). For example, the energy emitting device 40 (e.g., using the processor 44) may turn on the generator 41 to a default drive level.

The energy emitting device 40 may wait for the system to stabilize (step 83). This wait period is also shown as the first rectangle-shaped region in each of FIGS. 9A and 9B, respectively.

The energy emitting device 40 may measure the instantaneous power levels (step 84). For example, the energy emitting device 40 (e.g., using the power sensing unit 42) may measure instantaneous forward power levels and instantaneous reflected power levels. Signals indicative of these measurements are provided to the processor 44 of the energy emitting device 40. Instantaneous power levels may be measured throughout the time interval Δt (and throughout the treatment period as a whole). The frequency at which the power sensing unit 42 measures the instantaneous power levels may be configured and/or adjusted throughout the treatment period.

Next, the energy emitting device 40 may determine one or more instantaneous received power values (step 85). For example, the energy emitting device 40 (e.g., using processor 44) may determine an instantaneous received power value by subtracting an instantaneous reflected power value from an instantaneous forward power value. In some embodiments, the energy emitting device 40 may determine only one instantaneous received power value per time interval Δt. In some embodiments, the energy emitting device 40 may determine multiple instantaneous received power values per time interval Δt.

Continuing with the FIG. 10 example, the energy emitting device 40 may determine, during the first time interval Δt of 1 ms, a first instantaneous received power value of 45 W, a second instantaneous received power value of 50 W, and a third instantaneous received power value of 55 W. For example, throughout the first time interval Δt, the energy emitting device 40 may have measured three instantaneous forward power levels and three instantaneous reflected power levels (e.g., at three times during the first time interval Δt). Next, the energy emitting device 40 may determine each respective instantaneous received power value based on a difference between an instantaneous forward power level and a corresponding instantaneous reflected power level. As a specific example, a first instantaneous forward power level may be measured as 50 W and a second instantaneous reflected power level may be measured as 5 W. In this example, the energy emitting device 40 may subtract the reflected power level (5 W) from the forward power level (50 W) to determine that the first instantaneous received power value is 45 W.

In some embodiments, the one or more instantaneous received power values may be determined based on other measured electrical parameters (e.g., voltage, current, etc.). A description of these embodiments is provided elsewhere herein.

The energy emitting device 40 may determine whether the instantaneous received power values are within a normal operating range (step 86). For example, the energy emitting device 40 may be configured with lower bound and upper bound values that define the boundaries of a normal operating range. A normal operating range may refer to a range of values in which the energy emitting device 40 is determined to be operating at a normal or expected level. In this case, the energy emitting device 40 may compare each respective instantaneous received power value with the lower bound and upper bound values to determine whether the respective instantaneous received power values are within the normal operating range. To provide an example, if the energy emitting device 40 is not properly connected to the biological tissue or short circuited, the instantaneous received power values will be zero. In this example, the energy emitting device 40 may determine that the instantaneous received power values of zero are not within a normal operating range.

While step 86 involves using instantaneous received power values to determine whether operating conditions are normal, it is to be understood that this is provided by way of example. In practice, the energy emitting device 40 may determine whether operating conditions are normal using other related electromagnetic energy delivery parameters, including a VSWR parameter, a return loss parameter, a reflection coefficient parameter, and/or the like.

If the instantaneous received power values are not within the normal operating range, the energy emitting device 40 may terminate the treatment procedure, pause the treatment procedure, alert an operator such as a physician, and/or adjust one or more operating conditions of the generator 41 (step 87). Operating conditions may be adjusted by adjusting a state of the generator 41 and/or adjusting a drive level.

In some embodiments, the energy emitting device 40 may determine one or more actions to perform based on the degree to which the instantaneous received power values deviate from the normal operating range. For example, the energy emitting device 40 may reference a data structure that associates data identifying particular actions (e.g., terminate treatment procedure, pause treatment procedure, alert operator, etc.) with particular ranges of operating conditions. To provide a specific example, if the instantaneous received power values are zero, the energy emitting device 40 may simply generate an alert for the operator. However, if the instantaneous received power values are extremely high, the energy emitting device 40 may pause or terminate the treatment procedure and may then generate an alert for the operator.

If the instantaneous received power values are within the normal operating range, the energy emitting device 40 may determine a period of additional time needed to deliver a remaining amount of electromagnetic energy during a PWM period T (step 88). In order to deliver the appropriate total amount of electromagnetic energy over the treatment period, the energy emitting device 40 has to deliver x amount of electromagnetic energy each PWM period. That is to say, if the total amount of electromagnetic energy over the treatment period is 1000 mJ, and the treatment period is split into ten PWM periods, then ˜100 mJ of electromagnetic energy should be delivered during each PWM period (assuming all other operating conditions are constant). However, in practice, operating conditions and other factors are not constant. Consequently, at various times within a given PWM period T, the actual amount of electromagnetic energy delivered may be ahead of, or behind, the amount of electromagnetic energy that should have been delivered at that moment in time within the PWM period T. To address this, the energy emitting device 40 may, at a given moment in time within a PWM period T, determine a remaining amount of electromagnetic energy that has to be delivered during the PWM period T, and may further determine an additional period of time needed to deliver that remaining amount of energy.

In some embodiments, the energy emitting device 40 may determine the period of additional time using Equation 3 below:

$$t 1 = \frac{{E}_{r}}{P} \qquad ( 3 )$$

In Equation 3, t1 is the additional period of time, Eᵣ is the remaining electromagnetic energy to be delivered over a given PWM period T, and P is an instantaneous received power value. First, the energy emitting device 40 may determine the remaining amount of electromagnetic energy Eᵣ that needs to be delivered over the PWM period T. Because electromagnetic energy has yet to be delivered, this amount is also the total amount of electromagnetic energy that is to be delivered over the PWM period T. Using the FIG. 10 example, the energy emitting device 40 may determine the remaining energy Eᵣ (at the beginning of the first time interval Δt) by multiplying the PWM period T of 10 ms by the treatment energy delivery value of 35 W to determine a remaining electromagnetic energy Eᵣ of 350 mJ. Next, at the beginning of the first time interval Δt of 1 ms, the energy emitting device 40 may use Equation 3 to determine the additional period of time t1, where the remaining energy Eᵣ is 350 mJ and where the instantaneous received power P is 45 W. The value of 45 W represents the instantaneous received power value determined at the beginning of the first time interval Δt. Using these values, the energy emitting device 40 may determine that the additional period of time t1 is 7.78 ms (e.g., 350 mJ/45 W).

Next, the energy emitting device 40 determines whether the period of additional time t1 exceeds the PWM period T (step 89). Using the example of FIG. 10, the period of additional time is 7.78 ms and the PWM period T is 10 ms. That is to say, the period of additional time (7.78 ms) does not exceed the PWM period T of 10 ms.

If the period of additional time t1 exceeds the PWM period T, the energy emitting device 40 may terminate the treatment procedure, pause the treatment procedure, alert an operator such as a physician, and/or adjust one or more operating conditions of the generator 41 (step 87).

If the period of additional time t1 does not exceed the PWM period T, then the energy emitting device 40 may also determine whether the period of additional time t1 exceeds the time interval Δt (step 90). If the period of additional time t1 does not exceed the time interval Δt, the energy emitting device 40 may, after the period of additional time t1 has lapsed, adjust operating conditions by powering off the generator 41 (step 92). Because the period of additional t1 does not exceed the time interval Δt, the energy emitting device 40 may select the duration of the period of additional time t1 as the time during which to deliver the remaining electromagnetic energy Eᵣ. After the period of additional time has lapsed, the energy emitting device 40 (e.g., using the processor 44) may instruct the generator 41 to power off such that power is temporarily no longer delivered to the biological tissue. This may cause the energy emitting device 40 to switch from PWM high-time to PWM low-time (step 92, step 93 and FIG. 9A). The generator 41 may remain in a powered off state until the PWM period T lapses.

Next, the energy emitting device 40 may determine whether the treatment period has lapsed (step 94). For example, the energy emitting device 40 may track a total time during which the treatment procedure has been performed and may compare the total time during which the treatment procedure has been performed to the treatment period that identifies the total treatment time. If the total time during which the treatment procedure has been performed exceeds the total period, the energy emitting device 40 may end the treatment (step 95). If the total time during which the treatment procedure has been performed does not exceed the treatment period, the energy emitting device 40 may repeat steps 82-94 (e.g., beginning with adjusting an operating condition by powering on the generator 41, as is shown in step 82).

If the period of additional time t1 exceeds the time interval Δt, the energy emitting device 40 may continue to deliver power until the time interval Δt lapses (step 91). Because the period of additional t1 exceeds the time interval Δt, the energy emitting device 40 may select the duration of the time interval Δt as the time during which to deliver the remaining electromagnetic energy Eᵣ. After delivering power during the time interval Δt, the energy emitting device 40 may instruct the power sensing unit 42 to measure the instantaneous power levels (step 84). This is important because if power had continued to be delivered past the time interval Δt, the power variance in the instantaneous received power values may exceed a threshold tolerance level. This increases the likelihood of the delivered power causing the patient to feel pain and increases the likelihood of unacceptable power monitoring and/or calculation errors.

While not described in the steps above, the energy emitting device 40 may, at the end of the first time interval, determine an actual amount of electromagnetic energy delivered for the first time interval Δt. Continuing with the FIG. 10 example, the energy emitting device 40 may multiply the instantaneous received power value of 45 W by the first time interval Δt value of 1 ms to determine that the actual amount of electromagnetic energy delivered (during the first time interval Δt) is 45 mJ.

One of ordinary skill in the art can appreciate that while a single instantaneous received power value of 45 W was used in this computation, that in other embodiments, multiple instantaneous received power values may be considered, an average between multiple instantaneous received power values may be considered, etc. For example, during the first time interval Δt, the energy emitting device 40 determined instantaneous received power values of 45 W, 50 W, and 55 W. If an average instantaneous received power were used to determine the actual (or estimated) amount of electromagnetic energy delivered during the first time interval Δt, then the energy emitting device 40 may multiply the average instantaneous received power value of 50 W by the first time interval Δt value of 1 ms to determine that the actual (or estimated) amount of electromagnetic energy delivered during the first time interval is 50 mJ.

One or more of the steps of FIG. 10 are repeated throughout the PWM period T. Continuing with the FIG. 10 example, during a second time interval Δt of 1 ms, the energy emitting device 40 may determine, at the beginning of the second time interval Δt, a new instantaneous received power value of 60 W. The energy emitting device 40 may also determine, at the beginning of the second time interval Δt, a remaining amount of electromagnetic energy Eᵣ, to be delivered over the PWM period T. Using the FIG. 10 example, the energy emitting device 40 may subtract the energy delivered during the first time interval (45 mJ) from the total electromagnetic energy delivered over the PWM period T (350 mJ) to determine that the remaining amount of electromagnetic energy Eᵣ, to be delivered over the PWM period T is 305 mJ. Next, the energy emitting device 40 may determine the period of additional time t1′ to be 5.08 ms (e.g., 305 mJ/60 W). The energy emitting device 40 may determine that the additional period of time 5.08 t1′ is greater than the second time interval Δt duration of 1 ms, thereby causing the energy emitting device 40 to continue delivering power for the duration of 1 ms. During this time, the energy emitting device 40 continues to measure instantaneous power levels. Toward the end of the second time interval Δt, the energy emitting device 40 has now determined two additional instantaneous received power values of 70 W and 80 W.

The energy emitting device 40 may now determine a power variance between the three instantaneous received power values determined during the second time interval Δt (e.g., 60 W, 70 W, and 80 W). Using the Pₘₐₓ−Pₘᵢₙ equation discussed in FIG. 6, the energy emitting device 40 determines that the power variance is 20 W. Next, the energy emitting device 40 determines that the change in the power variance is 33.3% (e.g., by dividing the power variance of 20 W by the Pₘᵢₙ of 60 W). The energy emitting device 40 then compares the change in the power variance (33.3%) to the upper bound threshold tolerance level (25%). Because the power variance (33.3%) exceeds the upper bound threshold tolerance level (25%), the energy emitting device 40 can reduce the duration of the time interval Δt from 1 ms to 0.5 ms. The energy emitting device 40 may also determine the actual electromagnetic energy delivered during the second time interval Δt (e.g., 60 W*1 ms=60 mJ). This value of 60 mJ can be used in computations made during the third time interval Δt (which is now 0.5 ms) to determine the remaining amount of electromagnetic energy Eᵣ₂ that has to be delivered throughout the PWM period T.

In some embodiments, the duration of the PWM period T may be less than the duration of the time interval Δt. In this case, the calculated PWM on and off time (power duty) may not be changed for every PWM period T, and the calculated PWM power duty may also apply to subsequent PWM period(s).

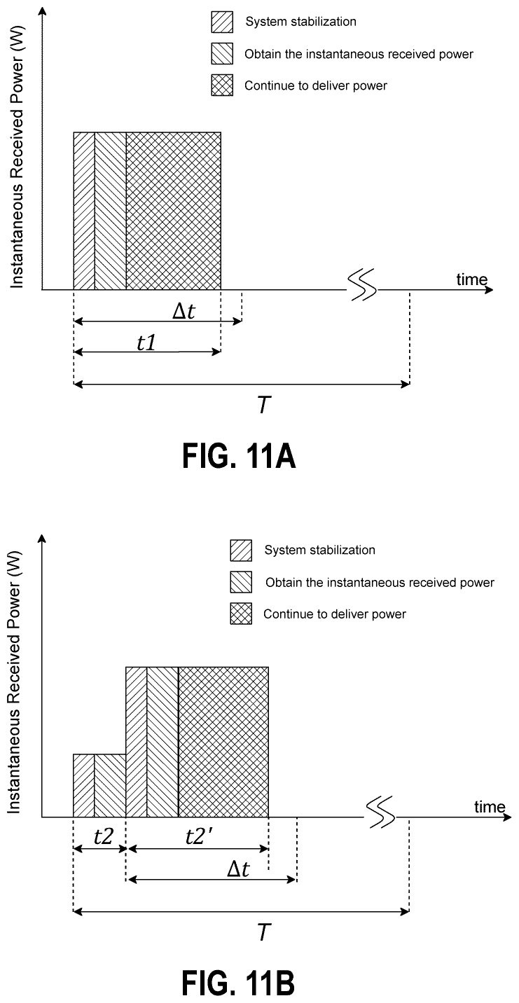

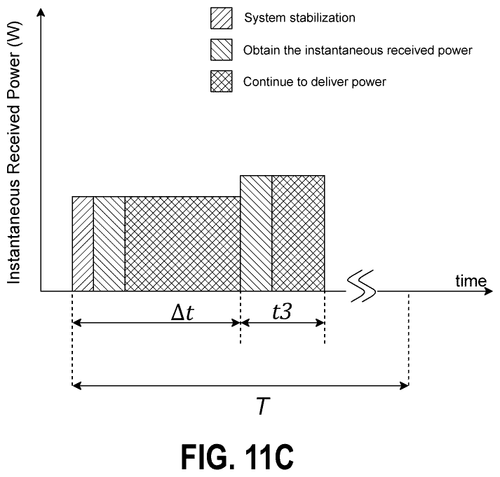

Now referring to FIGS. 11A, 11B, 11C, and 12, the energy emitting device 40 may use dynamic PWM with drive level control to deliver electromagnetic energy to biological tissue of a patient. Dynamic PWM involves varying a width or a duration of electrical pulses during the treatment procedure. To control the electromagnetic energy delivered to the biological tissue (e.g., using drive level control), the energy emitting device 40 may adjust (e.g., increase or decrease) a drive level of the generator 41.

FIG. 12 illustrates an example process for using dynamic PWM with drive control to deliver electromagnetic energy to biological tissue. Additional details for one or more of the steps described in FIG. 12 can be found in the description for corresponding steps of FIG. 10.

The energy emitting device 40 may receive treatment configuration data (step 100). An example will be provided throughout the description of FIG. 12 (referred to hereafter as the FIG. 12 example). In this example, the configured values are as follows: the PWM period T is 10 ms, the treatment energy delivery value is 35 W, the upper bound threshold tolerance level is 0.25 (25%), the lower bound threshold tolerance level is 0.05 (5%), and the initial duration of each respective time interval Δt is 1 ms.

The energy emitting device 40 may select a set of time intervals to use throughout the treatment period (step 101). Next, the energy emitting device 40 may turn on the generator 41 using a default power level (step 102). As the energy emitting device 40 switches from PWM low-time to PWM high-time, energy emitting device 40 may wait for the system to stabilize, which can be seen in step 103 and in FIG. 12.

The energy emitting device 40 may measure the instantaneous power levels (step 104). For example, the energy emitting device 40 (e.g., using the power sensing unit 42) may measure instantaneous forward power levels and instantaneous reflected power levels. Signals indicative of these measurements are provided to the processor 44 of the energy emitting device 40. Instantaneous power levels may be measured periodically throughout the time interval Δt (and throughout the treatment period as a whole).

Next, the energy emitting device 40 may determine one or more instantaneous received power values (step 105). For example, the energy emitting device 40 (e.g., using processor 44) may determine an instantaneous received power value by subtracting an instantaneous reflected power value from an instantaneous forward power value. Continuing with the FIG. 12 example, the energy emitting device 40 may determine, during the first time interval Δt of 1 ms, a first instantaneous received power value of 55 W, a second instantaneous received power value of 50 W, and a third instantaneous received power value of 45 W.

The energy emitting device 40 may determine whether the instantaneous received power values are within a normal operating range (step 106). If the instantaneous received power values are not within the normal operating range, the energy emitting device 40 may terminate the treatment procedure, pause the treatment procedure, alert an operator such as a physician, and/or adjust one or more operating conditions of the generator 41 (step 107).

If the instantaneous received power values are within the normal operating range, the energy emitting device 40 may determine a period of additional time needed to deliver a remaining amount of electromagnetic energy during a PWM period T (step 108). For example, the energy emitting device 40 may determine the period of additional time using Equation 3 as described in connection with FIG. 10. Using the FIG. 12 example, the energy emitting device 40 may determine the remaining energy Eᵣ (at the beginning of the first time interval Δt) by multiplying the PWM period T of 10 ms by the treatment energy delivery value of 35 W to determine a remaining electromagnetic energy Eᵣ of 350 mJ. Next, at the beginning of the first time interval Δt of 1 ms, the energy emitting device 40 may use Equation 3 to determine the additional period of time t1, where the remaining energy Eᵣ is 350 mJ and where the instantaneous received power P is 55 W. The value of 55 W represents the instantaneous received power value determined at the beginning of the first time interval Δt. Using these values, the energy emitting device 40 may determine that the additional period of time t1 is 6.36 ms (e.g., 350 mJ/55 W).

The energy emitting device 40 may determine whether the period of additional time t1 exceeds the PWM period T (step 109). Using the FIG. 12 example, the period of additional time t1 is 6.36 ms and the PWM period T is 10 ms. That is to say, the period of additional time t1 does not exceed the PWM period T. If the additional time t1 exceeds the PWM period T, the energy emitting device 40 may determine whether the maximum drive level on the electromagnetic energy generator 41 has been reached (step 116). If the maximum drive level has been reached, then the energy emitting device 40 may continue to deliver power until the PWM period T has lapsed (step 118) and/or may maintain the power delivery during the time interval Δt (step 111).

After the PWM period T has lapsed, the energy emitting device 40 may terminate the treatment procedure, pause the treatment procedure, alert an operator such as a physician, and/or adjust one or more operating conditions of the generator 41 (step 119). If the maximum drive level on the electromagnetic energy generator 41 has not been reached, the energy emitting device 40 may increase the drive level, which can be seen in step 117 and in FIG. 11B. Next, the energy emitting device 40 will repeat step 103 (e.g., using a PID control technique or a similar technique). If the additional time t1 does not exceed the PWM period T, then the energy emitting device 40 may determine whether the additional time t1 exceeds the time interval Δt (step 110).

In another case, as illustrated in FIG. 11C, the system may divide the power delivery into multiple intervals within the PWM period T, rather than a single continuous delivery. This allows for finer control of the power delivered to match the required treatment energy while staying within the predefined tolerance levels.

In some embodiments, if the additional time t1 exceeds the time interval Δt, the energy emitting device 40 may allow the power to be delivered during time interval Δt (step 111) and may instruct the power sensing unit 42 to continue measuring instantaneous power levels (step 104). Additionally, or alternatively, if the additional time t1 exceeds the time interval Δt, the energy emitting device 40 may determine whether the maximum drive level on the electromagnetic energy generator 41 has been reached (step 116). If the maximum drive level on the electromagnetic energy generator 41 has been reached, the energy emitting device 40 may maintain the power delivery during the time interval Δt (step 111), after which the energy emitting device 40 may then direct the power sensing unit 42 to continue measuring the instantaneous power levels (step 104). If the maximum drive level on the electromagnetic energy generator 41 has not been reached, the energy emitting device 40 may increase the drive level (step 117) and may repeat step 103.

If the additional time t1 does not exceed the time interval Δt, the energy emitting device 40 may terminate the power delivery from the electromagnetic energy generator 41 precisely after the period of additional time t1 has passed. This may cause the energy emitting device 40 to switch from PWM high-time to PWM low-time, as can be seen in step 112, step 113, and in FIG. 11A. The energy emitting device 40 may also track a total time during which the treatment procedure has been performed and may compare the total time during which the treatment procedure has been performed to the treatment period that identifies the total treatment time (step 114). If the total time during which the treatment procedure has been performed exceeds the total period, the energy emitting device 40 may end the treatment (step 115). If the total time during which the treatment procedure has been performed does not exceed the treatment period, the energy emitting device 40 may repeat steps 102-114 (e.g., beginning with adjusting an operating condition by powering on the generator 41, as is shown in step 102).

In an alternative embodiment, if the additional time t1 exceeds the time interval Δt, the energy emitting device 40 may allow the power to be delivered for a continuous time of Δt (step 111), after which the energy emitting device 40 may then direct the power sensing unit 42 to obtain the instantaneous received power again (step 104). Comparing t1 to the time interval Δt may be essential because once the continuous power delivery time exceeds the time interval Δt, the variation in the instantaneous received power may exceed the tolerance, and if the power calculation continues with the value obtained in the previous Δt, it may lead to unacceptable power monitoring or calculation errors.

One or more of the steps of FIG. 12 are repeated throughout the PWM period T. Continuing with the FIG. 12 example, during a second time interval Δt of 1 ms, the energy emitting device 40 may determine, at the beginning of the second time interval Δt, a new instantaneous received power value of 25 W. The energy emitting device 40 may also determine, at the beginning of the second time interval Δt, a remaining amount of electromagnetic energy Eᵣ, to be delivered over the PWM period T. Using the FIG. 10 example, the energy emitting device 40 may subtract the energy delivered during the first time interval (55 mJ) from the total electromagnetic energy delivered over the PWM period T (350 mJ) to determine that the remaining amount of electromagnetic energy Eᵣ, to be delivered over the PWM period T is 295 mJ. Next, the energy emitting device 40 may determine the period of additional time t1′ to be 11.8 ms (e.g., 295 mJ/25 W). The energy emitting device 40 may determine that the additional period of time t1′ of 11.8 ms is greater than the PWM period T of 10 ms. That is to say, even if 100% power duty is delivered, the power delivered will be insufficient to meet the amount specified by the treatment energy delivery value. To address this, the energy emitting device 40 may increase the drive level of the generator 41 to make the generator 41 output a higher value of power. Power may continue to be delivered for a duration of 1 ms, and during this time, the energy emitting device 40 continues to determine instantaneous received power values.

Toward the end of the second time interval Δt, the energy emitting device 40 may determine a power variance between the three instantaneous received power values determined during the second time interval Δt (e.g., 25 W, 23 W, and 21 W). Using the Pₘₐₓ-Pₘᵢₙ equation discussed in FIG. 6, the energy emitting device 40 determines that the power variance is 4 W. Next, the energy emitting device 40 determines that the change in the power variance is 19% (e.g., by dividing the power variance of 4 W by the Pₘᵢₙ of 21 W). The energy emitting device 40 then compares the change in the power variance (19%) to the upper bound threshold tolerance level (25%). Because the power variance (19%) is between the upper bound threshold tolerance level (25%) and the lower bound threshold tolerance level (5%), the energy emitting device 40 can maintain the same time interval duration (e.g., 1 ms). The energy emitting device 40 may also determine the actual electromagnetic energy delivered during the second time interval Δt (e.g., 25 W*1 ms=25 mJ). This value of 25 mJ can be used in computations made during the third time interval Δt to determine the remaining amount of electromagnetic energy Eᵣ₂ that has to be delivered throughout the PWM period T.

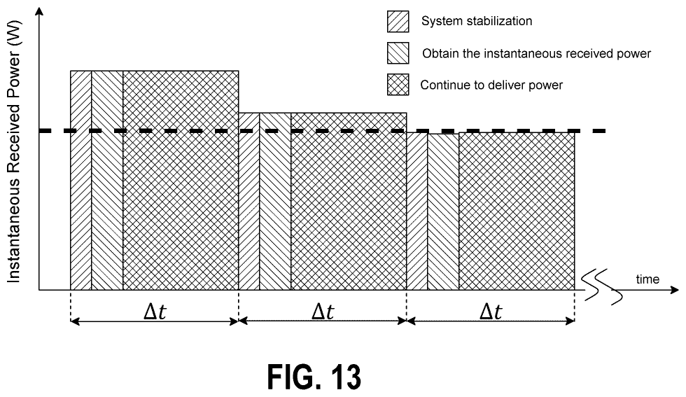

Now referring to FIG. 13 and FIG. 14, the energy emitting device 40 may use dynamic amplitude modulation to deliver electromagnetic energy to biological tissue of a patient. The amplitude modulation can be achieved by adjusting one or more operating conditions, such as by adjusting the drive level on the generator 41.

The energy emitting device 40 may receive treatment configuration data (step 120). The energy emitting device 40 may select a set of time intervals to use throughout the treatment period (step 121). Next, the energy emitting device 40 may turn on the generator 41 using a configured power level (step 122). As the energy emitting device 40 switches from PWM low-time to PWM high-time, energy emitting device 40 may wait for the system to stabilize, which can be seen in step 123 and in FIG. 13.

The energy emitting device 40 may measure the instantaneous power levels (step 124). For example, the energy emitting device 40 (e.g., using the power sensing unit 42) may measure instantaneous forward power levels and instantaneous reflected power levels. Signals indicative of these measurements are provided to the processor 44 of the energy emitting device 40. Instantaneous power levels may be measured periodically throughout the time interval Δt (and throughout the treatment period as a whole).

Next, the energy emitting device 40 may determine one or more instantaneous received power values (step 125). For example, the energy emitting device 40 (e.g., using processor 44) may determine an instantaneous received power value by subtracting an instantaneous reflected power value from an instantaneous forward power value. The energy emitting device 40 may determine whether the instantaneous received power values are within a normal operating range (step 126).

If the parameter(s) is (are) not in the normal range, the energy emitting device 40 may terminate or pause the treatment and alert the physician or system operator to take appropriate action. The energy emitting device 40 may also decrease the power duty to less than 40%, more preferred less than 20%, even more preferred less than 10%, most preferred less than 5% (step 127). Otherwise, the energy emitting device 40 continues to check if the treatment period has lapsed (step 128). If yes, the energy emitting device 40 may end the treatment (step 129). If no, the energy emitting device 40 determines whether the instantaneous received power values exceed the treatment power delivery value (step 130).

If the instantaneous received power is equal to the treatment energy delivery value, the energy emitting device 40 may not adjust the drive level on the electromagnetic energy generator 41 but may proceed to step 124 after the time interval Δt has lapsed (not shown in FIG. 13). If the instantaneous received power is greater than the treatment energy delivery value, the energy emitting device 40 checks if the drive level on the electromagnetic energy generator 41 has reached its minimal setting (step 131). If no, energy emitting device 40 may keep the power delivery until the time interval Δt has lapsed (step 133).

The energy emitting device 40 may then decrease the drive level on the electromagnetic energy generator 41 (step 134), and wait for system to stabilize before taking another instantaneous forward and reflected power measurement (step 123). If the drivel level has already reached its minimal setting, the energy emitting device 40 may terminate or pause the treatment and alert the physician or the system operator to take appropriate action. The energy emitting device 40 may also decrease the power duty to less than 40%, more preferred less than 20%, even more preferred less than 10%, most preferred less than 5% (step 132). If the instantaneous received power is smaller than the treatment energy delivery value, the energy emitting device 40 checks if the drive level on the electromagnetic energy generator 41 has reached its maximum setting (step 135). If no, the energy emitting device 40 may keep the power delivery until the time interval Δt has lapsed (step 136).

The energy emitting device 40 may then increase the drive level on the electromagnetic energy generator 41 (step 137), and wait for system to stabilize (step 123) before taking another instantaneous forward and reflected power measurement (step 124). If the drive level has already reached its maximum setting, the energy emitting device 40 may terminate or pause the treatment and alert the physician or the system operator to take appropriate action. The energy emitting device 40 may also decrease the power duty to less than 40%, more preferred less than 20%, even more preferred less than 10%, most preferred less than 5% (step 138).

Without wishing to be bound by theory, in one treatment session the processor 44 may direct the energy emitting device 40 to deliver energy substantially equal to the energy delivery value by switching the operating conditions of the electromagnetic energy generator 41 between dynamic pulse width modulation (dPWM) without control over the drive level on the electromagnetic energy generator 41, dynamic pulse width modulation (dPWM) with control over the drive level on the electromagnetic energy generator 41, and the dynamic amplitude modulation (dAM).

In some embodiments, and as shown in FIG. 12 and FIG. 14, the energy emitting device 40 may use proportional integral derivative (PID) control algorithm to increase or decrease the drive level on the electromagnetic energy generator 41 to achieve: 1) faster instantaneous received power convergence to the treatment energy delivery value; 2) minimal overshoot or deviation of the instantaneous received power from the treatment energy delivery value. In particular, the PID control program may be included in the processor 44. The processor 44 may dynamically adjust the values of the critical PID control parameters including Kp, Ki, and Kd to achieve fast instantaneous received power convergence to the treatment energy delivery value and minimal undesired overshoot or deviation of the instantaneous received power from the treatment energy delivery value. According to other embodiments of the present invention, other control algorithms as known by one with ordinary skills in the art may be employed to accelerate convergence of the instantaneous received power to the treatment energy delivery value, as well as to minimize overshoot or deviation of the instantaneous received power from the treatment energy delivery value.

The foregoing disclosure provides illustration and description but is not intended to be exhaustive or to limit the embodiments to the precise form disclosed. Modifications and variations may be made in light of the above disclosure or may be acquired from practice of the embodiments.

As used herein, the term “component” is intended to be broadly construed as hardware, firmware, and/or a combination of hardware and software.

Certain user interfaces have been described herein. A user interface may include a graphical user interface, a non-graphical user interface, a text-based user interface, etc. A user interface may provide information for display. In some embodiments, a user may interact with the information, such as by providing input via an input component of a device that provides the user interface for display. In some embodiments, a user interface may be configurable by a device and/or a user (e.g., a user may change the size of the user interface, information provided via the user interface, a position of information provided via the user interface, etc.). Additionally, or alternatively, a user interface may be pre-configured to a standard configuration, a specific configuration based on a type of device on which the user interface is displayed, and/or a set of configurations based on capabilities and/or specifications associated with a device on which the user interface is displayed.

It will be apparent that systems and/or methods, described herein, may be implemented in different forms of hardware, firmware, and/or a combination of hardware and software. The actual specialized control hardware or software code used to implement these systems and/or methods is not limiting of the embodiments. Thus, the operation and behavior of the systems and/or methods were described herein without reference to specific software code—it being understood that software and hardware can be used to implement the systems and/or methods based on the description herein.

No element, act, or instruction used herein should be construed as critical or essential unless explicitly described as such. Also, as used herein, the articles “a” and “an” are intended to include one or more items and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method of delivering electromagnetic energy from an energy emitting device for treating biological tissue as part of a treatment procedure, comprising:
generating electromagnetic energy with a generator operating under one or more operating conditions for delivering the electromagnetic energy to the biological tissue;
delivering the electromagnetic energy through the energy emitting device to the biological tissue during a plurality of time intervals that extend over a treatment period of time that represents a duration of the treatment procedure;
while delivering a portion of the electromagnetic energy in a time interval of the plurality of time intervals, determining one or more instantaneous received power values based on measured electrical parameters;
determining, at least once during the time interval, a power variance based at least in part on the one or more instantaneous received power values;
updating a duration of one or more time intervals, of the plurality of time intervals, based on determining that a change in the power variance satisfies a threshold tolerance level; and
adjusting, during the time interval, at least one of the one or more operating conditions of the generator, adjusting at least one of the one or more operating conditions causing an amount of the electromagnetic energy delivered to the biological tissue to be equal to or substantially equal to an amount of electromagnetic energy identified by a treatment energy delivery value.

2. The method of claim 1, wherein determining an instantaneous received power value, of the one or more instantaneous received power values, comprises:
determining the instantaneous received power value based on the difference between a measured instantaneous forward power level and a measured instantaneous reflected power level.

3. The method of claim 1, wherein the one or more instantaneous received power values are a plurality of instantaneous received power values, and wherein determining the power variance comprises:
determining the power variance based on a difference between a minimum instantaneous received power value and a maximum instantaneous received power value of the plurality of instantaneous received power values.

4. The method of claim 1, wherein the one or more instantaneous received power values include only a single instantaneous received power value determined during the time interval, and wherein determining the power variance comprises:
determining the power variance based on a difference between the single instantaneous received power value determined during the time interval and a previously determined instantaneous received power value determined during a previous time interval of the plurality of time intervals.

5. The method of claim 1, wherein the threshold tolerance level is an upper bound threshold tolerance level; and wherein updating the duration of the one or more time intervals comprises:
reducing the duration of the one or more time intervals based on determining that the change in the power variance exceeds the upper bound threshold tolerance level.

6. The method of claim 1, wherein the threshold tolerance level is a lower bound threshold tolerance level; and wherein updating the duration of the one or more time intervals comprises:
increasing the duration of the one or more time intervals based on determining that the change in the power variance is less than the lower bound threshold tolerance level.

7. The method of claim 1, wherein the treatment period is further defined over a set of pulse width modulation (PWM) periods; and wherein the method further comprises:
determining, at the beginning of a first time interval of the plurality of time intervals, a remaining amount of the electromagnetic energy to deliver during a first PWM period of the set of PWM periods;
determining a period of additional time needed to deliver the remaining amount of the electromagnetic energy during the first PWM period;
comparing a duration of the period of additional time and a duration of an upcoming time interval of the plurality of time intervals;
selecting an amount of time during which to deliver the remaining amount of the electromagnetic energy during the first PWM period based on comparing the duration of the period of additional time and the duration of the upcoming time interval; and
wherein adjusting the one or more operating conditions comprises:
adjusting an operating condition, of the one or more operating conditions, to cause the generator to deliver, for the selected amount of time, the remaining amount of the electromagnetic energy during the first PWM period.

8. The method of claim 7, wherein selecting the amount of time during which to deliver the remaining amount of the electromagnetic energy over the first PWM period comprises:
selecting the period of additional time based on determining that the duration of the period of additional time does not exceed the duration of the upcoming time interval, or
selecting the upcoming time interval based on based on determining that the period of additional time exceeds the duration of the upcoming time interval.

9. The method of claim 7, wherein determining the period of additional time needed to deliver the remaining amount of electromagnetic energy during the first PWM period comprises:
determining the period of additional time using the following equation:
$$t 1 = \frac{{E}_{r}}{P} ,$$
where t1 is the additional period of time, Eᵣ is the remaining amount of the electromagnetic energy to deliver during the first PWM period, and P is based on the one or more instantaneous received power values.

10. The method of claim 1, wherein the one or more operating conditions include at least one of:
a state of the generator of the electromagnetic device, or
a drive level of the generator.

11. The method of claim 1, wherein each respective time interval, of the one or more time intervals, have a duration between 1 nanosecond (ns) and 10 second(s), preferably between 100 microseconds (us) to 10 milliseconds (ms).

12. The method of claim 1, further comprising:
determining that the one or more instantaneous received power values are not within a threshold range of values that represent normal operating conditions; and
performing one or more actions based on determining that the plurality of instantaneous received power values are not within the threshold range of values, wherein the one or more actions include at least one of:
pausing the treatment procedure,
terminating the treatment procedure,
adjusting an operating condition, of the one or more operating conditions, to reduce the amount of electromagnetic energy delivered by at least 60%, or
generating and transmitting an alert signal indicating that the energy emitting device is not operating under normal operating conditions.

13. An energy emitting device for treatment of biological tissue, the energy emitting device comprising:
a generator configured to generate electromagnetic energy and to operate under one or more operating conditions for delivering the electromagnetic energy to the biological tissue;
a power sensing unit configured to measure instantaneous power levels;
an electromagnetic energy delivery unit configured to convert the electromagnetic energy to energy forms permissible for the treatment of the biological tissue; and
a processor configured to:
instruct the generator to generate the electromagnetic energy, the electromagnetic energy to be delivered to the biological tissue during a plurality of time intervals that extend over a treatment period of time that represents a duration of the treatment procedure;
while a portion of the electromagnetic energy is delivered during a time interval of the plurality of time intervals, determine one or more instantaneous received power values based on measured electrical parameters;
determine, at least once during the time interval, a power variance based at least in part on the one or more instantaneous received power values;
update a duration of one or more time intervals, of the plurality of time intervals, based on determining that a change in the power variance satisfies a threshold tolerance level; and
adjust, during the time interval, at least one of the one or more operating conditions of the generator, adjusting at least one of the one or more operating conditions causing an amount of the electromagnetic energy delivered to the biological tissue to be equal to or substantially equal to an amount of electromagnetic energy identified by a treatment energy delivery value.

14. The energy emitting device of claim 13, wherein the processor, when determining an instantaneous received power value, of the one or more instantaneous received power values, is configured to:
determining the instantaneous received power value based on the difference between a measured instantaneous forward power level and a measured instantaneous reflected power level.

15. The energy emitting device of claim 13, wherein the threshold tolerance level is an upper bound threshold tolerance level; and wherein the processor, when updating the duration of the one or more time intervals, is configured to:
reduce the duration of the one or more time intervals based on determining that the change in the power variance exceeds the upper bound threshold tolerance level.

16. The energy emitting device of any of claim 13, wherein the threshold tolerance level is a lower bound threshold tolerance level; and wherein the processor, when updating the duration of the one or more time intervals, is configured to:
increase the duration of the one or more time intervals based on determining that the change in the power variance is less than the lower bound threshold tolerance level.

17. The energy emitting device of any of claim 13, wherein the treatment period is further defined over a set of pulse width modulation (PWM) periods; and wherein the processor is further configured to:
determine, at the beginning of a first time interval of the plurality of time intervals, a remaining amount of the electromagnetic energy to deliver during a first PWM period of the set of PWM periods;
determine a period of additional time needed to deliver the remaining amount of the electromagnetic energy during the first PWM period;
compare a duration of the period of additional time and a duration of an upcoming time interval of the plurality of time intervals; and
select an amount of time during which to deliver the remaining amount of the electromagnetic energy during the first PWM period based on comparing the duration of the period of additional time and the duration of the upcoming time interval; and
wherein the processor, when adjusting at least one of the one or more operating conditions, is configured to:
adjust an operating condition, of the one or more operating conditions, to cause the generator to deliver, for the selected amount of time, the remaining amount of the electromagnetic energy during the first PWM period.

18. The energy emitting device of any of claim 13, wherein the one or more operating conditions include at least one of:
a state of the generator of the electromagnetic device, or
a drive level of the generator.

19. The energy emitting device of any of claim 13, wherein the processor is further configured to:
determine that the one or more instantaneous received power values are not within a threshold range of values that represent normal operating conditions; and
perform one or more actions based on determining that the plurality of instantaneous received power values are not within the threshold range of values.

20. The energy emitting device of any of claim 13, wherein the power sensing unit comprises two directional couplers or one bi-directional coupler.
